SEQUENCE LISTING (32 sequences)

SEQ ID NO: 1 - DNA, 401 bp, Homo Sapiens
cattgtttctcccatgcagccctagccaggagtcctttcctggctccttctatccccgcagttcttttcttgaggaggacagatagggtttcttagggagttaggatgaagaaagcctaagtatgatgaattccccatccagcactgtgcccagcacatagcagacatccaataaaggtctgttaaatgaatgacacggamccgttgtttaagattttaataaaggtggggcaaagggttgaattggtcgaaaagtcctcaacgtcctgacgggactcccctgaagccaaggcagtttgagtccattcattattctgtcttggatcttaggcaaagccccgggccccttggagcagcaccagggttcccgacacccccaccgacagggtcgaacgtgcaca

SEQ ID NO: 2 - DNA, 201 bp, Homo Sapiens
gctcctgtaatcccagctactcaggaatctgaggcaagagaattgcttgaacttgggaggcggaggttgcagtgagccaagatcacgccactgcactccakcctgggcaacagagccaaactcagtctcaaaaaaaaaaaaaaaaaaaaagactttcatatagttctttccccagaggtctagggatgtggacctgtgagg

SEQ ID NO: 3 - DNA, 638 bp, Homo Sapiens
gctcctggggagagagcagaggtctaggaggccccatccaacccctgtggctcccgagtggcacgcgttcgaccccaagaccctacactcaccatggtcgataagtcttccgaacctctgagctccggacaggctctggaagtgctttagcttctttcctacacagcggcacccgccggcttccaggcttcacacttgtgaactcttcggctgcctctgacagtttatgtaatcctgggatgtcattcagttccctcctctgtgaaccctgatcacctccccacctctcttcctccgagccagcccccttcctttcctggaaatattgcaatgaaggatgtttcagggagggggrccgtaacaggaaggattctgcagggcatctagggttctgtgtctcctggcagtgtcctgatgactcaggcgccccaggcggtgaatgccctgttgactcgggagcctaagccttctctggtgggtgtgggaaaaggatgatcctcagtgccttaggccagtaccatactctgcactatccaaccccccaatccccctaccttatatcccagagaatctacttgattcatttctttgacttcttccttgtcttggtttatgttgatctcctgccaccaatccaa

SEQ ID NO: 4 - DNA, 742 bp, Homo Sapiens
ctgtgggcgtcatgggagtgtgtctggctggggaaatggggtgacttccacgacgcagcgcttgtgggtgtggcctccccactagcaggtgcatgtggaggggtcctggcccgtctaggcgggccgcaccttcatctctgacaccttgtagctatatttgtaccccttcgccgcactccagttgataccattggcatagctctcatggggtcccatgaggtagagaccattgaggtttgaagcatgacagtcggcgtaccaccaggctccytggaacttctcagcacaattcgaagaactcacatcattgtcttggtctttggtggagaagaagttgttgttgtggcccgttagagaattacctgctcacagaaaatgtggggtttgcagatgctggaaaaagccctgccactgtgagaccctcaccacatcctgcctctgagcagagccaacctcactttagcgacgcatgagccgagctacaggtgcacaaatgacccacccaggccttggaggaaggggaagcaggaggcaacctctggcaacctggtctggaaatctctctccacacctgaggcccctgctccagcaccacctgtttcctccagggctcccgcaaactgtcaggcctcgtgctctctcctggaatgaccttcctcctccagcctgggccgttctttcggtctttccccacgtccctggtggcccttggtaggtcaggacaccctcactgggggctttt

SEQ ID NO: 5 - DNA, 181 bp, Homo Sapiens
tgagaaattggagtctgagggaggctggcttctctagccctataagagggcaggcaccttttgaagcaaagaccagargagatggagctggacagagctgtgggggtcctgggcgctgccaccctgctgctctctttcctgggcatggcctgggctctccaggcggcagacacctgtccag

SEQ ID NO: 6 - DNA, 147 bp, Homo Sapiens
gacttatactgtctgtaatgatgttactgcctgtaacgatgctcacatttcctcctgcacaggagattccctgaygttccacaacaaccagtccttctccaccaaagaccaggacaatgatcttaacaccggaaattgtkctgtgat

SEQ ID NO: 7 - DNA, 980 bp, Homo Sapiens
acttgggaggctgcagaaggagaatcgcttgaaccccagaggcagaggttgcagtgagctgagatggcaccattgcactccagcctgggtgacaagagcaaaactccatctcaaaaaacaaaacaaaaaaagaaagcattgctgtccttcaacccagcaactccacctctaggaatgttttcctgaaaagaatagccaagggtgctcacaggaggtggtcattataatggcaaaaccctggagacaacttgatgcccaatagagtttaatccatcgcaggacccatacataacgaaatacaacacagccgtgaaatagatggtgcagactctgtgtctgacatggagagatggccagttttgccactaggagaaaaaggcacagagaattttgttggaaataaaatcaatcgaaagcaacttccagctttatctgactccctcaggcctctcctggcagacttgtatattttcttctcttgatgtaatctcctaattctgytcccagttagccaggctctaccccaacatgaacctggaactccagggatcagtgccctctgctccgctcctgaacttcagccctgggaatctgtctgtggacccctatatggagatagatgcctttgtgctcctgcccagctccagcaaggagcctgtcttccggctcagtgtggtaaggttcagagcctttgcaaatgctgtttccttttccccacatgcttttcccttttccccacatgcttttctccccactcaccacctgggtaattcctccaggtctcaacttaaatattacttcccgagagagggctttcttgctggcccttacccccaatctaccttacgccccttgccactttttcttgtaacaccccattatctttcttctcagcgtttactacaaagatactttgtagctgtctgcaggtgtatttgtttaatgcatgtatctctcactatgtgagtctgggctattgt

SEQ ID NO: 8 - DNA, 801 bp, Homo Sapiens
ttgaattcctgggtcacagggcatgcaactgtttaaacatttgccaattgccttccagaaaatttcaccaccagcaaggtttgagaaacactgttttcccattgggggaaacatttattttttaaataaacgagccaatatattttaaaaaagagagtttggttggtgccagatgtagcttcactctcaaccctgccgtctgctgggggtgagatctcaagggagctatgtggcttttttgaacctcagtttcctcatctgctaaaccaggccttcctagccgagctcttaagagactggctgagatgaggcaagatgaacagtgatgaatatggggtctttctgtggggagggtggacagggatgatcataggagtcaggcttcctcatccactgatctrggcagtgaaaactggcttctaagtatgaccttatgacttcagtgatgtcgcaagcaggttccagtccctgtgcctccccttcccgctcccaggaacccatccagcctcaggaactgcccccagccatcgagccttggctacttaagggacctgggcccaatccacagctgggacagtcctggcccactgcactgggaatctaggatgggggccttggccagagccctgccgtccatactgctggcattgctgcttacgtccaccccagaggctctgggtgccaaccccggcttggtcgccaggatcaccgacaagggactgcagtatggtaagaagccacatctgctggctggacttggcaaacccacgctccaggctgctctgggtacagtggggacag

SEQ ID NO: 9 - DNA, 543 bp, Homo Sapiens
cccttgctggctctgcaccactccctggctggcagcgcccctgttgtatgccagcctcttaacccaccttccttctccccagcacagatcatgtccctggtcactttcttcttcagcggggcataagccttctggcaggtgctgtggtcaacaatcgggatttcaatctagaacacaaagctgttattggtcctcatcccyggctgagaagcctctgctattctgacacccctgaagacggctcctctccactccatacctggacggacctgtgtctaccttagacttgatctaagagactgccccatgctacaggtgaaggaacagttgaatggagaatcaggaggcccgtgttctagggcagtcttggctgtgaagaccttggctgtgtgaacttgggcaagtcacatgccctctctgggcctcagatctctcttcagtaagatagaggtaataatacttgcctaggagatgtattagtcaataggacaatgtatacaaaaatgaagtttggttttcttttgtctcacccctactgtagga

SEQ ID NO: 10 - DNA, 801 bp, Homo Sapiens
cagctactcaggaggctgaggcaggaggattgcttaagcctggaaagatagaaattgcagtgagccgagatcacaccgctgcactccagtctgggcaacagagcaagaccctgtctcagaaaaaaaaaaaaaagggaaagaaaaaagaaagtaaacagaaaaccagagaagcaatagaagcagcaacagtagcagcagagggagttccgggcggttatggggcctgtagtcaccacacgaccgtactgctgcacgcgggcattggccggtcccaagatccatctttctgacaaactgcagtaaaggatttcaggggcaagtgaccctaaagaagaattcagaatatattaatttccttgtttatatcataaaatcactccccaaagctgtgctctcacagyagttcctattctagtgttttacgaggtgctcctgaagggggtggcagcacacatgactagtatctgcgtgtgctggggttggggggatgcctggcacatggacgcactgccccaccgcctcggtgcacgtctatgccacctaggttacgtatttcatcactcaacaggtattggcattttatgaatcagatggaacctttgcagcaatttataattttttttttgcttttcaccctaatacaaaaacatatcagatctcagccaggtgcggtggctcatgcctgtaatcccgacactcctggagaccaaggcgggtggatcacttgaggtgaggagttcaagaccagcctggccagcatggtgaaaccctgtctctactaaaaaaatacaaaaaattagc

SEQ ID NO: 11 - DNA, 801 bp, Homo Sapiens
cattttctgaggagctaagaaataccaaaaggttttatcaggtgtttgaactgtgatgttttaaaatagcctcacttgcctgtataggtagtcacccaccaattcatgtaatataatttttggttgacatcaaatatatacttcaaggcggtcataatttaagaaaaacacgattgacatttttacaaaggctctctacctatctgcttctttctccagtttgtggactatcagcccgcacaacaggagggcgtatatatggagggcaaaaggcaaaacctggtgattttccttggcaagtcctgatattaggtggaaccacagcagcaggtgcacttttatatgacaactgggtcctaacagctgctcatgccgtctatgagcaaaaacatgatgcatcygccctggacattcgaatgggcaccctgaaaagactatcacctcattatacacaagcctggtctgaagctgtttttatacatgaaggttatactcatgatgctggctttgacaatgacatagcactgattaaattgaataacaaagttgtaatcaatagcaacatcacgcctatttgtctgccaagaaaagaagctgaatcctttatgaggacagatgacattggaactgcatctggatggggattaacccaaaggggttttcttgctagaaatctaatgtatgtcgacataccgattgttgaccatcaaaaatgtactgctgcatatgaaaagccaccctatccaaggggaagtgtaactgctaacatgctttgtgctggcttagaaagtgggggcaagg

SEQ ID NO: 12 - DNA, 601 bp, Homo Sapiens
attgccagctggcttatgcctgttagcttagggaatgagaatgagtggaaacccaggtgtctgtaggagcttcctctttctctccatgtcctcttcgggtccatctgccacctgaatcccatctttgtatctgggcagctgattcccctccaggacctgttaactatcatttactcattggcaggccctgagctgaaagctggtgatccaaagaggaaggtctcaacctcagatcaacctcaaccttagtcaccaacccagcccagaattaactggagtttgcttccccttggtgttttasacaggcttgcctgggtaagcattttctctggaaatttcttactacgttggccctggcagagtgctaaatacctctctactttctggcaggaattccccatgttctgtgagtcaaaaaccactggcaattagaaaacttgccttgaatcctcctcccatttctcagtattaacctttcatttgttcttcagagctttcttccagctattgccccctaagaaacctcagcagcctagccccataggtctaagcctccaagtttccctttgaaaacaaagacagtctccactgcagatgaaca

SEQ ID NO: 13 - DNA, 601 bp, Homo Sapiens
tcctctggaaggtaaagaattgcagagacagaacagcccaacacgtacctggttcccccttttctcccttggtgccatcacgcccatctttgcctgggaagccgttgatgcctggagagctacaggcaatcactgcagggcaggtcttttgggcatcctcacaggtcacagtttctgagtaagacgctgccaccatactcaggagaaggagagggagtgatggaaacagggacatggtcctcaccttggtgtgagaaaactcagggaaggttaatctcagttaatgaacacatatttaccragcatgccctctgtcctacaatctgggtgcaggctatatagaaatagatgacccatccctggcctctagctggggatttggaaaagtaaacattccttgtgacactgcgtgactagtactttaacaaaggtaggcactatgatgagcagtggggatcctaaggaggggttcatctgtgcctagacacctggcgttgctgctggaagactataaacatgctttccgtggcagtgagaacaaatgggaccgtgcattgccagctggcttatgcctgttagcttagggaatgagaatgagtgg

SEQ ID NO: 14 - DNA, 601 bp, Homo Sapiens
gggctccaggctgggacccctccgtggggagctctgcggcaccacgctttgtgaatgggccctggggggaggttccgctgcctggggccccgatgcggggagccgcccttgaggcccccggagccacggaatagctgtcgcagggcgtggaacccgtgggcagccgcaggtgtgctcttgggggccaggacgccaggggcttccgaggtgttcacacctgcaaaccgccccgacctggcccccagactccttcctccgatttttggaaagaagtgcagctggcgctgccgcggaaggtgcrgtacaggcctgtggaaggagacccccagacgcagctgcaggacgacaaggaccccatgctgattcttcgaggccgagtccctgagggccgggccctggactcagaggtggacccggaccctgagggcgacctgggtatgcccgcccagccccactccccaactggagaagctcagcacagggcggagtgggggcaggcacagggcacagggcctggaggggctccaggtgttgaggactcttcccggcaccgggagcccctgcacggcctctgccctggaggtgctcggccctcggtctg

SEQ ID NO: 15 - DNA, 601 bp, Homo Sapiens
tgcccggcctgaattatacactttaaacgggtgtattttttatcgtttgtgaaattttttatggaaggtgaaattttttatggtttgtgaattatatatcaataaggttttgttaaaaaaaaacacaactaactacagattacatctaattgacagcatatttttactgccctgaatccaaaaagaaatgcccaggtatgggctaggggtgggcagagacacacacacttgtaatcatgtacacaactgggagtagtggggatgagtctgtggggaaaaacaggaattcagagtgctgagragcttcccttactgccctgcccacttccctcccctgggcattgctccctcagcctggattcacaaagtcaatgcctttgggagccaggaactaaattcttagtcaaagacacctccctcctgtccagttatgggtctcagggaggaaatggcaggtgggtggtttcattctttcagtttcagaaaatatctgtagacataacatatttcctctattaaacaaattctgaggtaaggggaaacagagagagacagtcagaacattataagtcgaaaagaatctcagcattcttagtttagg

SEQ ID NO: 16 - DNA, 201 bp, Homo Sapiens
acaacaatcttaaatatatcgcaaaattatatatctgagctttggacttctgacatcttatcactgtcaaaactgaggattttgataatttctcataatasaatccagtatcttgatatcagtgttttcaaattcaaccaggaattggaatacttggatttgtcccacaacaagttggtgaagatttcttgccaccctact

SEQ ID NO: 17 - DNA, 401 bp, Homo Sapiens
gaaaaccgaacacatcgctgacaacttggtgtatagacaaggccttcaaggaagtgccagaataatcaaaatctctgaagtccagctgaccctgtagcttcacgtttgaaattgagaaataccatacagttgtatgccaaaccagctggaggatcctaatgaaagaattccaagttgtttcaatgttgtttaaggtaagayttgataactttggatttgtttgaagtttcgccagaatacttaggaagtaagaacatttgttatcttctagcacacatttgatattagatagttccagatttgctacagtcttgactgacacatccaaaataaaatggaattctttgtttgtggggaacacaatgtgcagactctcagtgttaaagtcttgaaggccctca

SEQ ID NO: 18 - DNA, 2276 bp, Homo Sapiens
ttggggtacagttttattgataaactaacagagtagaaaaaatctaatggaatacccccaaatttaaaagagggcaagaaaagagagacaatagaacataaaacaaatgggacaagaataaagtacatagttgtcacagtcccttgggtgctgctgtaacaaaatacctgagactgggtaatttacaaagaacagaaatttatccattcatggttctggagtctgggaagtccaagattgaagggctgcatctggtgagggtcatctggctacattataacatgatggaaagcatcacatggtgagagagagcaagagaggacagaacttacttttataacaaactcattctcacaataaactgctcccttggtgataacattaatccatttatgagggtacagccttcataacctaatcacctcttaaaaggttccacctctcagctcgcagtgagccgagatcatgccactgcactccagcctgggtgacagagcgagactctgtctcaaaacaaacaaacaaacaaaaattccacctctcaacactgttgcattgaagatgaaatttccaatacatgagctttggggcacacattcaaaccatagcaatagttcaattactttaaagcccaaaatgtcagtaattatattaaatgcaatttaatacatcagtaactaaatgagatagactaaattttatgataatcagactaatattatcagactagacttcaaaacaacccaacaatcatatgctgtttacaagagacacatttatggatactaaaaggtataaagtgaacagatgcaaccatttatcaaaagaaagctgttgtggcaatattactattagacaaaatagaattaggaaaaaaatcataactgtagataaacgtttcactaagttaaaggtttcactgagaagaaataaaaattttgaatttataagtacttataacaacattaagaaactagaaaatagaaaaagaatagagccaaattgactgaactagaaaaatagaaaattcatagaaaaatccacaatcatagtgggagattttatcatacatctctcagtaactgatagaaaaaccagacaaaaatcaataaggataaagaaaatgcgaacacaatgtagttcacataaggaacctatactacttccaacagcagctgaaatcacagagcatacttaaaatctgaccatatactggctcatagagtaaattgcaatacattttaaagaatttaaatcatacagaatatgtcttctgaccataatgcaattaaagtagaaaagaacagaaaaacttatagaaaattctcatatatttggaaattagtaaatttctaaataacccatgggtcaaaaaataatcagaatgaaaatcagaaaatatttttaatgacttatgaaaaaaattacatataaaaacttgtcagatgcacctaaactgttgcttaaaggaaacttacaaccttaagtgcatatattagaaatgaagagtgacgaaaaatgaatgagcaagcaaataccttaaaaactggaaaaggaatagtaaaataaatccagagaaatcrgaacaggggaaataataatataagaatagaaattaatgtgctagaaaataggggatctatcaagtcagaagttgattttttgaaaatgctaataaaaatgatgaacctctggcaagacaataaaagctggtacaaataactcatctaaggaataaaattgaggacctctcttttgttaaatgacataagcaggaggccattatcctgagactgtctctacactttgagttcttacataataaactgcaacctaatttagtatactacataaacaaactgaaacctaacttaggagctttttttttgtaattaatagctaaatttccaccaaatcataaatagccaagctttagccaatcacaggcagccaactgatcagactatgcccaaacaaggtaaatgcctattacaccatacccaaataaggcagactcctagctgaagccaatcaggtgattactatgctcttgtgtttggcctataaaagctcactgctcacactgttggatgctctctgaaacttttgtgaatctgagtgctgcctgattcatgaatcattctttgctaaaataaactctgtcaaatttaatttgtctgaagtttctcttttaacagtgtttggtgttgcaagcaggatccaaaggagacctatagtgactcccaggagctcttagtgaccaagtgaaggtacct

SEQ ID NO: 19 - DNA, 625 bp, Homo Sapiens
taccaacctttagttttctggtctttaaaacgaagataatcgcatgtgttttgaattactgaattaagctaacttacgtaaaacgtctagtctagagcatcatcattacattccataaatgagagctatgatgaggattgaaaatgtggatagaatgaagatgataatatgtatctcaaagtattgtattgggattaaatgaactggcatttgtaaagcttttaggacagtgtctggaaagtagcaagtgcaatgtaagtttctgttaaataagaacctgatttaaaacaggaatattatgyatgctaagtcctagatagttctactgtaatatcctccaagcacttccaatccaacgtgtcaaaaaccaagctgtgagggaccattttgtggtggactgggcacaaactcttgtagactccatcaacacaccagtagagttaagataaaaacaacctccatgtcaaagctttgctctgagtattgagattattcatgtaaagattttagagcagtgcctgacacagagtaggtgctcaataaagtttagttccctggaaagttaatggtgtttcactttgacatagtcatccaaggtggaaatctctttgactttatttccctt

SEQ ID NO: 20 - DNA, 601 bp, Homo Sapiens
agctctgttgggatgtttttgagggactttctcatcttcaagttctgtatttgaatcataactatcttaattcccttccaccaggagtatttagccatctgactgcattaaggggactaagcctcaactccaacaggctgacagttctttctcacaatgatttacctgctaatttagagatcctggacatatccaggaaccagctcctagctcctaatcctgatgtatttgtatcacttagtgtcttggatataactcataacaagttcatttgtgaatgtgaacttagcacttttatcarttggcttaatcacaccaatgtcactatagctgggcctcctgcagacatatattgtgtgtaccctgactcgttctctggggtttccctcttctctctttccacggaaggttgtgatgaagaggaagtcttaaagtccctaaagttctcccttttcattgtatgcactgtcactctgactctgttcctcatgaccatcctcacagtcacaaagttccggggcttctgttttatctgttataagacagcccagagactggtgttcaaggaccatccccagggcacagaacctgatatgtacaa

SEQ ID NO: 21 - DNA, 1496 bp, Homo Sapiens
caatactttctgtttctttgcatggctaatttttttaaacaaataaattagagattcctttaatgccaagttacacaatcaaaaagataacaatctctttccatttgctttttgcaaggtttgtgttatttcagggcaaaacgtagagcaagtccactggcaaatctgggggctagtgattattttgttttgatttttatttttttaaatcagacattttaaacactacaatgaggtaactctagaaatcaaattctgtccccctttcccagaggtgttttatttatttatttatttattttttgacagggtctcattctgtcacccaggctggagtgcagtggtgtaatcacagctcattggagcctcgacttcctcggccccaaacaatcctcccactcagcctcccaagtagctgggactataggcacacgccaccatgcccagctaagttttgtattttttgtagagatggagtttcactatgttgcgcaggctggctttgaactcctggactcaagcaatccacctgccttgccctccccaagttctgggactataggcttaagccactgtgccgggcctccctttcccagggtgtattgttgcttcttgtggcttatagttgcttgtttaatgacttttctaaataatttttataaagattattctttgtcatgtgtggctattacattttctgtccagttagcttagtggtcagccagtaacttgagatttctttaaacatctggagcaaaaacaaaaaccaaataaactagcaaaagcaaaaacaaaaacacccctgatctttgcaggttggctgtgttggatcactttctcaatgcttagccagtctattcacagctctgctttagccttcactttctgcatgcatggaagctaaagatgagccagaggtgaagtttaggattttctcaggtctcctctgaacatgcgtccagccttgggcacgtgagtggacttctggactccctggtatatacagagatttcctcagccttttttctcccacatatctttytccatatatctttgcatatttcccagactttttgatgtgtctgctgcttgccccctctgttagcccttgctcaggtggctgcagctagtatgtttgcctttaattgtttacagcaaacaccagccagaaggctgttccagccctgagaaagttctaaggtgtacaaaacaaaggcaagcccctgagtgtgaatccctcaggcagcaaccagacaggtcaaaacacacaaccacaattctttgataacaaggcctatattgccccttctggcatgagcaagctatataactggttataaaaagtattacattaaattatggggacagaaatgactctactagtgaatgaagaaatcatttagattttatggttagtaagtggaggtttgagagtaaccatccgatctttattaatcttagtgtatattatatttctagaatttggactcatatca

SEQ ID NO: 22 - DNA, 601 bp, Homo Sapiens
aatttcttgggattgagtgctatgaagctgcaaaaattagatttgctgccaattgctcacttgcatctaagttatatccttctggatttaagaaattattatataaaagaaaatgagacagaaagtctacaaattctgaatgcaaaaacccttcaccttgtttttcacccaactagtttattcgctatccaagtgaacatatcagttaatactttagggtgcttacaactgactaatattaaattgaatgatgacaactgtcaagttttcattaaatttttatcagaactcaccagaggtycaaccttactgaattttaccctcaaccacatagaaacgacttggaaatgcctggtcagagtctttcaatttctttggcccaaacctgtggaatatctcaatatttacaatttaacaataattgaaagcattcgtgaagaagattttacttattctaaaacgacattgaaagcattgacaatagaacatatcacgaaccaagtttttctgttttcacagacagctttgtacaccgtgttttctgagatgaacattatgatgttaaccatttcagatacaccttttatacacatgctgtgtc

SEQ ID NO: 23 - DNA, 601 bp, Homo Sapiens
gacttggaaatgcctggtcagagtctttcaatttctttggcccaaacctgtggaatatctcaatatttacaatttaacaataattgaaagcattcgtgaagaagattttacttattctaaaacgacattgaaagcattgacaatagaacatatcacgaaccaagtttttctgttttcacagacagctttgtacaccgtgttttctgagatgaacattatgatgttaaccatttcagatacaccttttatacacatgctgtgtcctcatgcaccaagcacattcaagtttttgaactttacscagaacgttttcacagatagtatttttgaaaaatgttccacgttagttaaattggagacacttatcttacaaaagaatggattaaaagaccttttcaaagtaggtctcatgacgaaggatatgccttctttggaaatactggatgttagctggaattctttggaatctggtagacataaagaaaactgcacttgggttgagagtatagtggtgttaaatttgtcttcaaatatgcttactgactctgttttcagatgtttacctcccaggatcaaggtacttgatcttcacagcaataaa

SEQ ID NO: 24 - DNA, 1133 bp, Homo Sapiens
tgctgctgagccagccagatgcctggcactatggagcctgcctgccatgataccacccagagtgggcctggggggagcaggctggcagatgtgcagccttgcctcctcccagcagcaacaattcattaattcattcattcagccttcactcagaaataccctctctgtgcctgcccagagctgactgctgggtgtacataattcagcagatatcaagcacttactatgtgctgggcactgtactggatcctggggatgcagataaaagatcactgccctyaagaagctgacattccagcaggggaataagacgatatacaataaaccatgaaagatcaatgatccggtgtgctagcagttaaaaaatgttaggacaaagagaaacatagaccaggcaaaggagctcaggagtgccagatctggggtgggaggtttgtaagaaggctggatggccctgttgagagggtgacatgggagcagagacataatggaggcaaaggaggggtcatatgagacttgggggagttttcaggcagagggaacagcacatcccaaggccctgaggcaggagaatgcccaggggctacacagtcaactgcaagcaggccagtgggcctggagagcactcaggggaacagcgggtggcaggtcccatagggccttgttggccatctgtggactgtggctttgactccagagtcagccagggaggggtctgagcagaggagggacctagtgtgacttaggtttgaacagttccctctaggggctgaatgtgaccagcccctggagggcagagagcagggcaggacagccagaagggaggtggcacctgcatgggtctaggagagaggatgggggctggtggacatggggacggtgggctgtgggcacacccttcagcatggtggacccagcagaacttgctgaggggcccggggtcacccaaggctttgggcccgaggaatctgagtctcctcacctagatcagaaggagagtgggaagaactgatggggaggactggctggcggccctgctgggccagcacacacctggcctctaggagccccatctggagtgacgtggtgtgtgctctgatggggccccaggggctctgggaatgtggaggagg

SEQ ID NO: 25 - DNA, 201 bp, Homo Sapiens
gaggtttgtaagaaggctggatggccctgttgagagggtgacatgggagcagagacataatggaggcaaaggaggggtcatatgagacttgggggagtttycaggcagagggaacagcacatcccaaggccctgaggcaggagaatgcccaggggctacacagtcaactgcaagcaggccagtgggcctggagagcactca

SEQ ID NO: 26 - DNA, 2442 bp, Homo Sapiens
gttagacaactataaatgtcttaacttcttgttgaattcaaaggtcttgatattcagctgctgaattctgttatggcatagaatcaaagctttcaggttagatttcaatgtgccaagatacatactaattgcttcatgtatattatctcatttatttatcaccatactctgtgagggaagtactactactcccatttgatagatgaggaaattgaaggatatagaattctgaggactttgggagatgtgccaggcttcacgtggttggattagacgtagaacagaggtagttggagtactaagattttacgatattcattattcaagatatttattaagtacctattctgccagaaacttttctaggcactggggaagtagcagtgaacagaagagatttctgtttctacatagcttatattctagaaagaagttaagaaaagagagactgcaaacaaaacaatacgtttatatgttatgactcacaattccacatgcctggggaggcctcacaatcatggtggaaggcgaatgaggagcaaagtcatgtcttatgtggcagcaggcaagagagcatgtgcagggaacctcctctttataaaaccaccagatctcatgagacttattcactatcatgagaacagcatgggaaagacccgcccccatgattcaattacttcccaccaggtccctcccacaacatgtgggaattatgggagctacagttcaagatgagatttgggtggggaccaatatcaggcagtgatattgtcttgaaggaaaaataactaagataagggaaacagagagagactggggagtgattttcttttttatagaatgatcaaaaatggcctccctgatagtgatcttcatgcagaaatctaaaggggccaaagaagcaagacacttgttgtctggggaaagagtattttggagagagagagaacagcaagtacaaatttaaagatcctgatgtgcttggaaaatgtgaggaagacctggagtggagttaggggacggaatcagagaagaggtcggatcttataagaacttggcaattttcctctgactgaaataagaaaccagcagaagatttagagtccaggaatgrcaggttctgacatacattttaaagattcctctgtttgaggcaacctatagaatgggagaaaatttttgcaatctgtccatctgacaaagggctaatacccagaatctataaagaacattaaaaaatttacaagaaaaaaacaaccccatcaaaaagtgggtgaaggatatgaacaggcacttctcaaaagaagacatttatgcagccaacaaacatatgaaaaaaagctcatgatcactggtcattagagaaatgcaaatcaaaaccacaatgagataccatctcatgcctgttagaatggtgatcattaaaaagtcaggagacaacaggtgctggagagggtgtagagaaataggaacgcttttacactgttggtgggggtgtaaattagctcaacaattgtggaagacaatgtggcgattcctcaaggatctagaactagaaatactatttgatcctgcaatgccattactgggtatctatccaaaggattataaatcattctactataaagatacatgcacacacgtgtttattgcagcactattcacaacagcaaagacttggaaccaacccaaatgcccatcaatgatagactggataaagaaaatatggcacatatacaccatggaatactatgcagccataaaaaagaatgtgttcatgtcctttgcagggacatggatgaatctggaaaccatcattctcagcaaactaatacaagaacagaaaaccaaacaccacatgttctcactcataactgggagttgaacaatgagaacacattgacacacaccggggcctgtcagggggtggggggctaggggaggaatagcattaggagaaatacctaatgtagatgatgggctgatgggtgcagcaaaccaccatggcatgtgtatacctatgtaacaaacatgcacgttccgcacatgtaccccagaacttacagtataaaataaataaataaataaataaacaaacaaacaaataaaaagattcctctgtttgttgggtggaaatgactgggagccacaaggctggcagggaagactccttgggacactatagcgtaacttgtgccagagagaatgtatcaatggaagtggagagaagtggttagattctaatttcaaattagtaaacaatttaactctacgtgtccccacaccaggctctctgaaggcaccaagagcaggtgataagttagtgggggagtttttctgcttccatttgtatgtatctgttacacattgaccctcaacctccctcagacaggtcactcccatagacaagcagagggagcaaaagatatgctcaacagaaag

SEQ ID NO: 27 - DNA, 1016 bp, Homo Sapiens
tataccctttcttgaaactcattcacttgtcttaattctcctcctacttccttgacctctattttgctatatagtcttcatattctctgctgtggtcccaacatactggtaactttcaaatccatatttccctcaagtttctctcttcttagctctagtttcctatattcaattatcttaatataaatatatggatatcccacaggcttttcacactcagcatgtccataacagagctcatcatcttccctctaaaatttgctttgcctacaaaattcccagaatcagaaaatactactaccatctatccagtcaccaaaaccagagacctggtgttgaatatggggggtctctgggttaaatttgtctcccagatctaattctcacaactatatctacattcaagtttatctacctagatcactctcctgctctagccaagcaaaattattagcagttttacatgattcttctgtgcctttgcacgtgtttttctcaagaatgcgctccagctgtttccctttggctggcattcttcttcctacttattcttgaagaccagttcaaggtgcacttactctgtgaaatgtcttttcccacttttgtccccatactttaattatacctacctcaaaaattataaatacctcaaatacagttatttcttaaatgtgtatttgttgatttttaagtctgtcttctctactgaaccttaagttcattgagagcrcagattattatccccagctgctaacataatttgaacctttgttcgcctatttgggccttggttctatctgtaaaacaggaacaaaaaaaaaatctactctgcaaaattattgtgaggatcaaagataccgtctgcctagtaagaagccagaacaaagtaggtgcatataaattagtgaatgttatcattactgataaataagtgggtgaccaagcatctgatgaagagagggattagactgacaatgtgaacattcatgttgctttaaaggaattgcaagtagctc

SEQ ID NO: 28 - DNA, 601 bp, Homo Sapiens
atgtcacacctggaaatcctaggtttgagtggggcaaaaatacaaaaatcagatttccagaaaattgctcatctgcatctaaatactgtcttcttaggattcagaactcttcctcattatgaagaaggtagcctgcccatcttaaacacaacaaaactgcacattgttttaccaatggacacaaatttctgggttcttttgcgtgatggaatcaagacttcaaaaatattagaaatgacaaatatagatggcaaaagccaatttgtaagttatgaaatgcaacgaaatcttagtttagaamatgctaagacatcggttctattgcttaataaagttgatttactctgggacgaccttttccttatcttacaatttgtttggcatacatcagtggaacactttcagatccgaaatgtgacttttggtggtaaggcttatcttgaccacaattcatttgactactcaaatactgtaatgagaactataaaattggagcatgtacatttcagagtgttttacattcaacaggataaaatctatttgcttttgaccaaaatggacatagaaaacctgacaatatcaaatgcacaaatgccacaca

SEQ ID NO: 29 - DNA, 601 bp, Homo Sapiens
atcggttctattgcttaataaagttgatttactctgggacgaccttttccttatcttacaatttgtttggcatacatcagtggaacactttcagatccgaaatgtgacttttggtggtaaggcttatcttgaccacaattcatttgactactcaaatactgtaatgagaactataaaattggagcatgtacatttcagagtgttttacattcaacaggataaaatctatttgcttttgaccaaaatggacatagaaaacctgacaatatcaaatgcacaaatgccacacatgcttttccckaattatcctacgaaattccaatatttaaattttgccaataatatcttaacagacgagttgtttaaaagaactatccaactgcctcacttgaaaactctcattttgaatggcaataaactggagacactttctttagtaagttgctttgctaacaacacacccttggaacacttggatctgagtcaaaatctattacaacataaaaatgatgaaaattgctcatggccagaaactgtggtcaatatgaatctgtcatacaataaattgtctgattctgtcttcaggtgcttgcccaaaagt

SEQ ID NO: 30 - DNA, 801 bp, Homo Sapiens
caggagcctgtgcccctgggaccccacgttatgcctgtctgcctgccaaggcttgagcctgaaggcccggccccccacatgctgggcctggtggccggctggggcatctccaatcccaatgtgacagtggatgagatcatcagcagtggcacacggaccttgtcagatgtcctrcagtatgtcaagttacccgtggtgcctcacgctgagtgcaaaactagctatgagtcccgctcgggcaattacagcgtcacggagaacatgttctgtgctggctactacgagggcggcaaagacacgtgccttggagatagcggtggggcctttgtcatctttgatgacttgagccagcgctgggtggtgcaaggcctggtgtcctgggggggacctgaagaatgcggcagcaagcaggtctatggagtctacacaaaggtctccaattacgtggactgggtgtgggagcagatgggcttaccacaaagtgttgtggagccccaggtggaacggtgagctgacttacttcctcggggcctgcctcccctgagcgaagctacaccgcacttccgacagcacactccacattacttatcagaccatatggaatggaacacactgacctagcggtggcttctcctaccgagacagcccccaggaccctgagagcagagtgtggtatagggaaaaggcctccagcagagacctgtgttcctgagcttgtcaagtctctttccctgtctggggctactctacccagtatacatgcaggagctcaaccaaggctttgtgcaattccacactcct

SEQ ID NO: 31 - DNA, 601 bp, Homo Sapiens
attagagctattttggactccttgacaatcttttaggtctagcaaactacctaaccccctcccaacgacatcatttcatagtcttcatatccacatgttgatatctatgatctcaactgagtcccaccccctgatttcataattgaacacactgatgccccacccctccacccctcaacccccacccctgagaaagtaagtgacttacccaaggtcacatgcaagctggcctatagtcactgagcccagttctgtttccagcctaatgtcccttccattcctcccacccctgatgacccaygatctcttagtgacacttcagggagattcatcaaaagcatcagacaatatgtagtgtaaccaagtagaaaactgtcctgaatcacatgcagatcattcctgggccacgtagttcctgtttttatgcagatttcctctgagccttggctaactcccagggaagcgcacatatcagatctgctatatattggcagaactttggcttagaacacatctcgaatgctttccattgaacattggcagccttgaagaaacatggctagaaaattattccaggaggcctaattgctctgtgacactt

SEQ ID NO: 32 - DNA, 601 bp, Homo Sapiens
ggttccttgtttactttcacaacatttaaaatcattagaatacttggatctcagtgaaaatttgatggttgaagaatacttgaaaaattcagcctgtgaggatgcctggccctctctacaaactttaattttaaggcaaaatcatttggcatcattggaaaaaaccggagagactttgctcaytctgaaaaacttgactaacattgatatcagtaagaatagttttcattctatgcctgaaacttgtcagtggccagaaaagatgaaatatttgaacttatccagcacacgaatacacagygtaacaggctgcattcccaagacactggaaattttagatgttagcaacaacaatctcaatttattttctttgaatttgccgcaactcaaagaactttatatttccagaaataagttgatgactctaccagatgcctccctcttacccatgttactagtattgaaaatcagtaggaatgcaataactacgttttctaaggagcaacttgactcatttcacacactgaagactttggaagctggtggcaataacttcatttgctcctgtgaattyctstccttcactcaggagcagcaagca

(19) United States
(12) Patent Application Publication
(10) Pub. No.: US 2011/0020803 A1
(43) Pub. Date: Jan. 27, 2011

(54) MARKERS FOR THE PROGNOSIS OF LUNG FUNCTION AND COLONIZATION WITH AN INFECTIOUS AGENT

(76) Inventors: Lieve Nuytinck, Drogen (BE); Els De Meester, Antwerpen (BE)

Correspondence Address: NIXON & VANDERHYE, PC, 901 NORTH GLEBE ROAD, 11TH FLOOR, ARLINGTON, VA, 22203, US

(21) Appl. No.: 12/733,907

(22) PCT Filed: Sep. 29, 2008
(86) PCT No.: PCT/EP2008/063022

§ 371 Date: Sep. 23, 2010

Related U.S. Application Data
(60) Provisional application No. 60/960,458, filed on Oct. 1, 2007.

(30) Foreign Application Priority Data
Sep. 28, 2007 (EP) 07019135.8

(51) Int. Cl. C12Q 1/68

(52) U.S. Cl. 435/6

(57) ABSTRACT

The present invention provides a method and kit for determining the risk for impaired lung function and/or the susceptibility for colonization with an infectious agent. The method and kit comprise determining the presence or absence of one or more nucleic acid variants in a gene or a combination of genes.

### SUMMARY OF THE INVENTION

The present invention provides a method and kit for determining the risk for impaired lung function and/or the susceptibility for colonization with an infectious agent. The method and kit comprise determining the presence or absence of one or more nucleic acid variants in a gene or a combination of genes.

### BACKGROUND OF THE INVENTION

Cystic fibrosis (CF) is a common monogenic recessive hereditary disease caused by mutations in the Cystic Fibrosis Transmembrane Regulator (CFTR) gene of which the encoded protein is acting as a chlorine channel. Mutations in the gene give rise to defects in the amino acid sequence or the expression level of the protein, thereby impairing or destroying its function. The gene is approximately 250 kbases and more than thousand mutations have been described, so far. The most frequent mutation is deltaF508 (about 70% in the Western population). In Caucasians, one in 25 individuals carries at least one mutation and about 1 in 2500 are affected. Some decades ago CF patients had a very poor live expectancy; most of them died during childhood. Nowadays, due to early prevention of the complicating factors many reach the adult stage.

The severity of the disease is linked to the type of mutations present. Some (like deltaF508) are considered to be severe; many others are considered as mild mutations. However, among those with so-called severe mutations, individuals are found that have mild symptoms only; and seriously ill patients are found among those with mild mutations. This indicates that other factors, possibly also genetic factors, play a role in the disease severity. Therefore the impact of genes involved in the different innate immunity pathways need to be investigated.

Poor lung function, and even more important, rapidly declining lung function, and Pseudomonas aeruginosa and/or Staphylococcus colonisation are indicators for a bad disease prognosis in CF patients. CF patients are particularly susceptible to respiratory infections from bacteria, and, in particular P. aeruginosa. For example, a chronic respiratory infection, particularly an infection of the lung by P. aeruginosa, accounts for almost 90% of the morbidity and mortality in CF. Progressive loss of pulmonary function over many years due to chronic infection with mucoid P. aeruginosa is common in subjects suffering from CF. Smith et al. (1996) reported defective bacterial killing by fluid obtained from airway epithelial cell cultures of CF patients, and suggested that this phenomenon was due to the inhibition of an unidentified antimicrobial factor resulting from increased levels of sodium chloride in the airway epithelial fluid.

It is key to take measures as early as possible in childhood to prevent or delay lung function deterioration and infections of the respiratory tract. Knowledge of the genetic constitution of the patient may provide timely information on the patient's susceptibility towards infections and poor lung function. Using this information, patient treatment may be guided with respect to frequency of follow-up, preventive antibiotic administration, etc. As a result CF patients can have a less severe disease course, an increased life-expectancy, and an overall better quality of life.

The present invention provides a means for producing novel prognostic indicators for the susceptibility for colonization with an infectious agent as well as for the deterioration of the lung function.

### SUMMARY OF THE INVENTION

In a first embodiment, the present invention provides a method or kit for determining the risk for impaired lung function in a subject, comprising determining the presence of at least one nucleic acid variant in a gene selected from the group consisting of the nucleic acid variants as given in Table 4, and/or, determining the presence of a combination of nucleic acid variants in one or more genes, whereby the combination SNP1/SNP2 is selected from the group consisting of the nucleic acid variants as given in Tables 5 and 6. More specific, the presence of one, or the combined presence of the at least two nucleic acid variants is indicative of the risk for deterioration or decline of the pulmonary function. In a particular embodiment, the subject is diagnosed with cystic fibrosis.

In a further embodiment, the invention encompasses a method or kit for determining the susceptibility for colonisation with an infectious agent, comprising determining the presence of at least one nucleic acid variant in a gene selected from the group consisting of the nucleic acid variants as given in Tables 7 and 9, and/or, determining the presence of a combination of nucleic acid variants in one or more genes, whereby the combination SNP1/SNP2 is selected from the group consisting of the nucleic acid variants as given in Tables 8 and 10. More specific, the presence of one, or the combined presence of the at least two nucleic acid variants is indicative of susceptibility for colonization with an infectious agent, especially of the respiratory tract. In a specific embodiment, the infectious agent is a bacterium. In a more specific embodiment, the infectious agent is Pseudomonas aeruginosa or Staphylococcus aureus.

In a preferred embodiment, the nucleic acid variant determined in the method or kit of the present invention is a single nucleotide polymorphism (SNP). In a further aspect of the method or kit, it is determined whether said nucleic acid variant in the gene is present in 0, 1 or 2 copies, wherein the heterozygous or homozygous presence of one or of the combination of the at least two nucleic acid variants is indicative of the predisposition for impaired lung function and/or susceptibility for colonization with an infectious agent.

Methods for determining the presence or absence of the nucleic acid variants of the present invention include but are not limited to DNA or RNA hybridization, sequencing, PCR, primer extension, multiplex ligation-dependent probe amplification (MLPA), oligonucleotide ligation assay (OLA) and/or restriction site analysis. Preferably, the step of determining the presence of the nucleic acid variant is performed in vitro in a biological sample obtained from said subject.

The kit according to the present invention comprises one or more reagents for detecting the presence of at least one nucleic acid variant in a gene selected from the group consisting of the nucleic acid variants as given in Tables 4, 7 or 9, and/or, for detecting the presence of a combination of nucleic acid variants in one or more genes, whereby the combination SNP1/SNP2 is selected from the group consisting of the nucleic acid variants as given in Tables 5, 6, 8 and 10. In a specific embodiment, the reagents are primers or probes.

The present invention also encompasses a diagnostic kit comprising:
one or more allele specific primer and/or one or more oligonucleotide probe for detecting the presence of at least one nucleic acid variant as given in Tables 4, 7, or 9, and/or,
one or more allele specific primer and/or one or more oligonucleotide probe for detecting the presence of a combination of nucleic acid variants in one or more genes, whereby the combination SNP1/SNP2 is selected from the group consisting of the nucleic acid variants as given in Tables 5, 6, 8, and 10.

In a specific embodiment, the kit further comprises a table indicating the link between the SNP or SNP combinations and the risk for deterioration of lung function and/or susceptibility for colonization with an infectious agent.

### FIGURE LEGENDS

The present description including the Examples is to be understood in conjunction with the following Figures intended to illustrate the invention without implying any limitation of the invention to the specific embodiments described therein.

FIG. 1: Fragments of the CD14 gene comprising a nucleic acid variant.

FIG. 2: Fragment of the FCN1 gene comprising a nucleic acid variant.

FIG. 3: Fragments of the FCN2 gene comprising a nucleic acid variant.

FIG. 4: Fragments of the LBP gene comprising a nucleic acid variant.

FIG. 5: Fragment of the MASP1 gene comprising a nucleic acid variant.

FIG. 6: Fragments of the MASP2 gene comprising a nucleic acid variant.

FIG. 7: Fragments of the MBL2 gene comprising a nucleic acid variant.

FIG. 8: Fragment of the Sigirr gene comprising a nucleic acid variant.

FIG. 9: Fragments of the TLR1 gene comprising a nucleic acid variant.

FIG. 10: Fragments of the TLR2 gene comprising a nucleic acid variant.

FIG. 11: Fragment of the TLR4 gene comprising a nucleic acid variant.

FIG. 12: Fragment of the TLR5 gene comprising a nucleic acid variant.

FIG. 13: Fragments of the TLR6 gene comprising the nucleic acid variant.

FIG. 14: Fragments of the TLR9 gene comprising a nucleic acid variant.

FIG. 15: Fragments of the TLR10 gene comprising a nucleic acid variant.

FIG. 16: Fragment of the MASP3 gene comprising a nucleic acid variant.

FIG. 17: Fragment of the MASP1-3 gene comprising a nucleic acid variant.

### DETAILED DESCRIPTION OF THE INVENTION

Unless defined otherwise, all technical and scientific terms used herein have the same meaning as commonly understood by one of ordinary skill in the art. In any case the meaning of a term should not be limited to less than would be commonly understood by one of ordinary skill. The examples are only illustrative and not limiting.

### DEFINITIONS

Unless otherwise specified, a “gene” refers not only to the coding sequence, but to all sequences that are part of that gene: the introns and exons, the regulatory regions including the promoter region and possible other regulatory sequences, such as 5′UTR, 3′UTR or sequences further up- or downstream.

As used herein, the term “wild-type” sequence refers to the reference sequence. The reference nucleic acid and protein sequences indicated in the current invention are derived from NCBI (National Center for Biotechnology Information; http://www.ncbi.nlm.nih.gov/) and indicated by their respective accession number (Table 1), as is well known to the person skilled in the art. The nomenclature for the nucleotide and amino acid changes as used herein is generally accepted and recommended by den Dunnen and Antonarakis (2000). Frequent updates of the nomenclature for the description of sequence variations are provided on the web-site of the Human Genome Variation Society.

Accordingly, the nucleotide numbering of the coding DNA and RNA reference sequence is as follows:
nucleotide+1 is the A of the ATG-translation initiation codon
there is no nucleotide 0
the nucleotide 5′ of the ATG-translation initiation codon is −1.

To avoid confusion, the nucleotide number is preceded by “g.” when a genomic or by “c.” when a cDNA reference sequence is used. Substitutions are designated by “>”.

The term “nucleic acid” refers to a single stranded or double stranded nucleic acid sequence and may consist of deoxyribonucleotides or ribonucleotides, nucleotide analogues or modified nucleotides. There is no limitation in length. A nucleic acid that is about 100 nucleotides or less in length is often also referred to as an oligonucleotide.

The term “nucleic acid variant” or “polymorphism” as used in the present invention refers to a position comprising one or more nucleotides in the nucleic acid sequence which differs relative to a reference nucleic acid sequence.

The most simple nucleic acid polymorphism is a polymorphism affecting a single nucleotide, i.e. a single nucleotide polymorphism or SNP. The term “Single nucleotide polymorphism (SNP)” refers to the variation of a single nucleotide. This includes the replacement of one nucleotide by another and deletion or insertion of a single nucleotide. Typically, SNPs are biallelic markers although tri- and tetra-allelic markers also exist. For example, SNP A\C may comprise allele C or allele A. Thus, a nucleic acid variant when referring to the SNP A\C may include a C or A at the polymorphic position. The SNPs of the present invention are disclosed in the NCBI database dbSNP, and are characterised by an “rs” number. Said rs number can be used to retrieve summary information on the known variation at the locus, a list of the specific reports that characterize a SNP, and links to other NCBI resources.

Nucleic acid polymorphisms further include any number of contiguous and/or non-contiguous differences in the primary nucleotide sequence of the nucleic acid under investigation relative to the primary nucleotide sequence of one or more reference nucleic acids. The term “polymorphic position” or “position” refers to the nucleic acid position at which a nucleic acid polymorphism arises. Nucleic acid sequences comprising at least one such polymorphism are referred to as “polymorphic nucleic acid sequences”, “polymorphic polynucleotides”, “polymorphic sequences” or the like. The polymorphism or nucleic acid variant can be an insertion, deletion, substitution, tandem repeat or similar.

The phrase “determining the presence”, e.g. of a marker or variant as used herein, refers to determining whether or not the relevant genetic, physiological and/or biochemical event, linked with the occurrence of a disease (state) is present. In practice, both the absence and the presence of a certain event can function as markers. Accordingly, reference to establish the presence of a nucleic acid variant for determining predisposition to impaired lung function or for determining susceptibility for colonization with an infectious agent generally encompasses determining whether the variant is present or absent in a sample. As such, this also includes the possible finding that the marker is not present in the sample, i.e. determining the absence of a nucleic acid variant. In both cases determining the presence of the marker can also be done indirectly, e.g., where the presence of a nucleic acid variant is linked to a disease (state), the occurrence of this marker can (besides the direct detection of the nucleic acid variant) also be done by determining the homozygous presence of the corresponding allele not comprising the nucleic acid variant. Similarly, allele specific oligonucleotide primers or probes for detecting the presence of a SNP can be specific for the allele where the SNP is not present.

The term “haplotype” means a particular pattern of sequential polymorphisms found on a single chromosome. As used herein, the term “allele” is one of several alternative forms of a gene or DNA sequence at a specific chromosomal location (locus). At each autosomal locus an individual possesses two alleles, one inherited from the father and one from the mother. The term “genotype” means the genetic constitution of an individual, either overall or at a specific locus, and defines the combination of alleles the individual carries. The term “homozygous” refers to having two of the same alleles at a locus; the term “heterozygous” refers to having different alleles at a locus.

The term “lung function” or “pulmonary function” as used herein refers to the functional status of the lungs. The primary instrument used in pulmonary function testing is the spirometer. The FEV1 test is a spirometric pulmonary function test routinely performed. The term “FEV1” (Forced Expiratory Volume in One Second) refers to the volume of air that can be exhaled during the first second of a forced exhalation, and is expressed as liters. It is a reflection of the flow of air in the large airways of the lung. “FVC” (Forced Vital Capacity) is the volume of air which can be forcibly and maximally exhaled out of the lungs until no more can be expired, after the patient has taken in the deepest possible breath. FVC is usually expressed in units called liters. “FEV1 Percent” (FEV1%) is the ratio of FEV1 to FVC. It indicates what percentage of the total FVC was expelled from the lungs during the first second of forced exhalation; this number is called FEV1%, % FEV1 or FEV1/FVC ratio.

The term “infectious agent” refers to viruses, bacteria, fungi, and parasites. Pathogenic bacteria include but are not limited to those species selected from the genera Streptococcus, Pneumococcus, Pseudomonas, Micrococcus, Enterococcus, Corynebacterium, and Staphylococcus. Staphylococcus aureus is typically regarded as the most pathogenic of the staphylococci. S. aureus is responsible for a wide range of infections, including soft tissue infections and potentially fatal bacteremias. By the term “colonization” is meant the presence and/or growth of the infectious agent in a subject during a period of at least 6 months. A specific site of colonization in the present context is the respiratory tract, including the nose and nasal passages, the throat and trachea, and the lungs.

As used herein, the term “Cystic fibrosis (CF)” refers to an inherited disease characterized by an abnormality in the body's salt, water- and mucus-making cells. It is chronic, progressive, and is usually fatal. In general, children with CF live into their 30s. In addition to a complete medical history and physical examination, diagnostic procedures for cystic fibrosis may include the following:
sweat (chloride) test—a test to measure the amount of chloride in the sweat. Higher than normal amounts of chloride may suggest cystic fibrosis,
blood tests—blood or cheek scraping cells can be tested for mutations in the CFTR gene. Other blood tests can asses infection, and involvement of certain organs,
chest x-rays—a diagnostic test which uses invisible electromagnetic energy beams to produce images of internal tissues, bones, and organs onto film,
pulmonary function tests—diagnostic tests that help to measure the lungs' ability to exchange oxygen and carbon dioxide appropriately,
sputum cultures—a diagnostic test performed on the material that is coughed up from the lungs and into the mouth. A sputum culture is often performed to determine if an infection is present,
stool evaluations—to measure stool fat absorption,
pancreatic function tests

The term “biological sample” means a tissue sample or a body fluid sample. A tissue sample includes (but is not limited to) buccal cells, a brain sample, a hair root, a skin sample or organ sample (e.g. liver). The term “body fluid” refers to all fluids that are present in the body including but not limited to blood, plasma, serum, synovial fluid, lymph, urine, saliva or cerebrospinal fluid. The biological sample may also be obtained by subjecting it to a pre-treatment if necessary, for example, by homogenizing or extracting. Such a pre-treatment may be selected appropriately by those skilled in the art depending on the biological sample to be subjected.

The “subject” on which the method of the present invention can be carried out can be any vertebrate animal, more particularly any mammalian animal, and is most particularly a human. It is envisaged that the methods of the invention can be applied to a non-human subject such as (but not limited to) a cow, a pig, a sheep, a goat, a horse, especially racing horses, a monkey, a rabbit, a dog, a cat, a mouse, a rat, a hamster or a primate or any laboratory test animal.

The present invention provides methods and tools for determining the risk for deterioration of the lung function in a subject. Poor lung function and rapidly declining lung function are indicators for a bad disease prognosis in CF patients. Furthermore, the present invention provides methods for determining the susceptibility of a subject for colonization with an infectious agent, specifically bacteria, and more specific with P. aeruginosa or S. aureus. It has been shown that certain nucleic acid variants, or combinations thereof, are associated with an increased, or alternatively a lower susceptibility for colonization with bacteria, especially P. aeruginosa or S. aureus. Many CF centres promote aggressive treatment regimens to eradicate first isolates of P. aeruginosa or S. aureus, and thus, prospectively identifying a “high risk” group of patients with CF could have substantial clinical benefit. Although antiinflammatory therapies to date have shown a reduction in the decline of lung function, the risk/benefit ratio does not favor long-term prednisolone treatment, and results from high-dose nonsteroidal antiinflammatory therapies have been associated with significantly enhanced side-effect profiles, including gastrointestinal bleeding. Therefore, the identification of a specific targeted antiinflammatory therapy based on individual patient genotypes may prove clinically useful.

In a first aspect, the present invention provides methods for determining the predisposition for an impaired lung function, and/or the risk of severe infection, based on the determination of the presence in a subject of one or a combination of at least two nucleic acid variants in a specific gene or combination of genes.

According to the present invention, determination of the occurrence of a polymorphism in a gene, possibly in combination with determination of the presence of another polymorphism in the same or another gene, allows the identification of a subject at risk of developing an impaired lung function with improved accuracy, and/or the identification of a subject at risk of colonization with an infectious agent, particularly with P. aeruginosa or S. aureus. More specific, the subject is diagnosed with cystic fibrosis.

The presence of a nucleic acid variant in a gene, or the presence of at least two nucleic acid variants in a gene or a combination of genes is associated with a (high) risk for impaired lung function and/or with susceptibility for colonization with an infectious agent, with an odds ratio of at least 3.00, 3.25, 3.26, 3.27, 3.28 or 3.29, preferably 3.5 and even more preferably 4.0, or more. Alternatively, the presence of a nucleic acid variant in a gene, or the presence of at least two nucleic acid variants in a gene or a combination of genes is associated with no or only a minor impairment or decrease of lung function and/or with protection for colonization with P. aeruginosa or S. aureus, with an odds ratio of less than 0.4, 0.39, 0.38, 0.37 or 0.36, and preferably 0.35, or less. Most particularly the upper and lower limit of the confidence interval for this odds ratio is <1 and >1, respectively.

According to a particular embodiment of the methods of the invention, the determination of the presence of one or more polymorphisms of a gene is ensured at the DNA level. Most particularly, the determination includes determining the occurrence of one variant allele of a gene (heterozygous for the variant allele) or the presence of two variant alleles of a gene (homozygous for the presence the variant allele). Determination of the occurrence of a variant allele at the DNA level is described in more detail below.

Additionally or alternatively, the determination of the presence of a variant allele can be performed at the protein level, based on quantitative expression of the expressed protein, qualitative assessment of the expressed protein (differences in protein sequence) and/or a functional assessment of the expressed protein (e.g. the ability to bind to GlcNAc). Methods for determining serum concentrations and altered function of a protein are described, for instance in Hummelshoj et al. (2005).

In a specific embodiment, the methods of the present invention involve the determination of a polymorphism in a gene, and more particularly, the determination of two or more polymorphisms in a gene or combination of genes selected from the group consisting of: mannose-binding lectin (MBL2), MBL-associated Serine Protease 1 and alternative splice variants thereof (MASP1, MASP1-3 and MASP3), MBL-associated Serine Protease 2 (MASP2), Ficolin 1 (FCN1), Ficolin 2 (FCN2), Complement component 1q Receptor 1 (C1qR1), Lipopolysaccharide-binding protein (LBP), Monocyte Differentiation Antigen CD14 (CD14), Single Immunoglobulin domain-containing IL1R-related protein (Sigirr), Toll-like Receptor 1 (TLR1), Toll-like Receptor 2 (TLR2), Toll-like Receptor 3 (TLR3), Toll-like Receptor 4 (TLR4), Toll-like Receptor 5 (TLR5), Toll-like Receptor 6 (TLR6), Toll-like Receptor 7 (TLR7), Toll-like Receptor 8 (TLR8), Toll-like Receptor 9 (TLR9), and Toll-like Receptor 10 (TLR10).

Sequence characteristics of the genes and associated proteins are given in Table 1. In a particular embodiment, the polymorphism can be detected in the corresponding cDNA or RNA sequence.

TABLE 1
gives for each gene of the present invention the number of
exons, the reference gDNA sequence (with the used version
between brackets), the reference mRNA sequence and the
reference protein sequence, as given by the NCBI.

| | Exons | gDNA | mRNA | Protein |
|---|---|---|---|---|
| FCN1 | 9 | NT_019501 | NM_002003 | NP_001994 |
| | | (NT_019501.13, | | |
| | | GI: 89030070) | | |
| FCN2 | 8 | NT_019501 | SV0: NM_004108 | NP_004099 |
| | | (NT_019501.13, | SV1: NM_015837 | NP_056652 |
| | | GI: 89030070) | | |
| TLR1 | 4 | NT_016297 | NM_003263 | NP_003254 |
| | | (NT_016297.15, | | |
| | | GI: 37539484) | | |
| TLR2 | 2 | NT_016354 | NM_003264 | NP_003255 |
| | | (NT 016354.18, | | |
| | | GI: 88977422) | | |
| TLR3 | 5 | NT_022792 | NM_003265 | NP_003256 |
| | | (NT_022792.17, | | |
| | | GI: 51464381) | | |
| TLR4 | 4 | NT_008470 | NM_003266 | NP_003257 |
| | | (NT_008470.18, | NM_138556 | NP_612566 |
| | | GI: 89030004) | NM_138554 | NP_612564 |
| | | | NM_138557 | NP_612567 |
| TLR5 | 6 | NT_021877 | NM_003268 | NP_003259 |
| | | (NT_021877.18, | | |
| | | GI: 88943807) | | |
| TLR6 | 1 | NT_016297 | NM_006068 | NP_006059 |
| | | (NT_016297.15, | | |
| | | GI: 37539484) | | |
| TLR7 | 3 | NT_011757 | NM_016562 | NP_057646 |
| | | (NT_011757.15, | | |
| | | GI: 89059526) | | |
| TLR8 | 3 | NT_011757 | NM_138636 | NP_619542 |
| | | (NT_011757.15, | NM _016610 | NP_057694 |
| | | GI: 89059526) | | |
| TLR9 | 2 | NT_ 022517 | NM_017442 | NP_059138 |
| | | SEQ-ID N: 9 | | |
| TLR10 | 3 | NT_016297 | NM_030956 | NP_112218 |
| | | (NT_016297.15, | | |
| | | GI: 37539484) | | |
| LBP | 15 | NT_011362 | NM_004139 | NP_004130 |
| | | (NT_011362.9, | | |
| | | GI: 51475129) | | |
| C1QR1 | 2 | NT_011387 | NM_012072 | NP_036204 |
| | | (NT_011387.8, | | |
| | | GI: 27501067) | | |
| MASP1 | 9 | NT_005612 | NM_001031849 | NP_001027019 |
| MASP1-3 | | (NT_005612.15, | NM_001879 | NP_001870 |
| MASP3 | | GI: 88966845) | NM_139125 | NP_624302 |
| MASP2 | 11 | NT_021937 | NM_ 006610 | NP_ 006601 |
| | | (NT_021937.18, | | |
| | | GI: 88942368) | | |
| MBL2 | 4 | NT_008583 | NM_000242 | NP_000233 |
| | | (NT_008583.16, | | |
| | | GI: 37551286) | | |
| CD14 | 2 | NT_029289 | NM_000591 | NP_000582 |
| | | (NT-029289.10, | | |
| | | GI: 37550092) | | |
| Sigirr | 10 | NT_035113 | NM_021805 | NP_068577 |
| | | (NT_035113.6, | | |
| | | GI: 51493057) | | |

Due to the presence of alternative splice forms, multiple transcript/protein entries can be provided for a single gene entry.

According to yet a further embodiment, the polymorphism in the gene is characterized as indicated in Table 2.

TABLE 2
gives NCBI's accession number for each SNP (rs number), the gene, the
region and position of the SNP, the nucleic acid polymorphism and the
amino acid change. It is to be understood that the nucleotide variant also
encompasses the complement nucleic acids.

| | | | Position in | | AA |
|---|---|---|---|---|---|
| Gene | Region | SNP | gDNA | Alleles | change |
| FCN1 | Exon 9 | rs1071583 | g.7919 | A/G | Q275Q |
| FCN2 | promotor | rs17514136 | g.−4 | A/G | na |
| | Exon 8 | rs17549193 | g.6359 | C/T | T236M |
| TLR 1 | promoter | rs5743551 | g.−7202 | A/G | na |
| | Exon 4 | rs5743611 | g.239 | G/C | R80T |
| | Exon4 | rs4833095 | g.743 | A/G | N248S |
| TLR 2 | promoter | rs1898830 | g.−15607 | A/G | na |
| TLR 4 | promoter | rs1927914 | g.−2026 | G/A | na |
| TLR 5 | Exon 6 | rs2072493 | g.1775 | A/G | N592S |
| TLR6 | Promotor | rs1039559 | g.−502 | C/T | na |
| | Exon1 | rs5743810 | g.745 | T/C | S249P |
| | Exon1 | rs3821985 | g.1083 | C/G | T361T |
| TLR9 | Promoter | rs187084 | g.−1486 | C/T | na |
| TLR9 | Promoter | rs5743836 | g.−1237 | T/C | na |
| TLR 10 | promoter | rs7694115 | g.−1883 | T/C | na |
| | promoter | rs11466645 | g.−992 | T/A | na |
| | Exon 3 | rs11096957 | g.721 | A/C | N241H |
| | Exon 3 | rs11096956 | g.1032 | G/T | P344P |
| MBL2 | Promoter | rs11003125 | g.−619 | G/C | na |
| MBL2 | 5′UTR | rs7095891 | g.−66 | C/T | na |
| CD14 | Promoter | rs3138078 | g.−1461 (v1) | G/T | na |
| CD14 | Promoter | rs2569190 | g.−260 (v1) | C/T | na |
| CD14 | IVS 2 | rs2563298 | g.1336 | G/T | na |
| MASP1-3 | Promoter | rs710474 | g.−2641 | T/C | na |
| MASP3 | Protease | rs850312 | g.55613 | G/A | L617L |
| | dom | | | | |
| MASP1 | IVS14 | rs3733001 | g.70465 | G/A | na |
| MASP2 | IVS8 | rs6695096 | g.12226 | G/A | na |
| MASP2 | Exon11 | rs1782455 | g.19741 | C/T | S493S |
| LBP | Promoter | rs2232578 | g.−205 | A/G | na |
| LBP | IVS9 | rs1780627 | g.22713 | T/C | na |
| Sigirr | Exon 9 | rs3210908 | g.3392 | A/G | Q312R |

v1 = variant 1
v2 = variant 2
na: not applicable

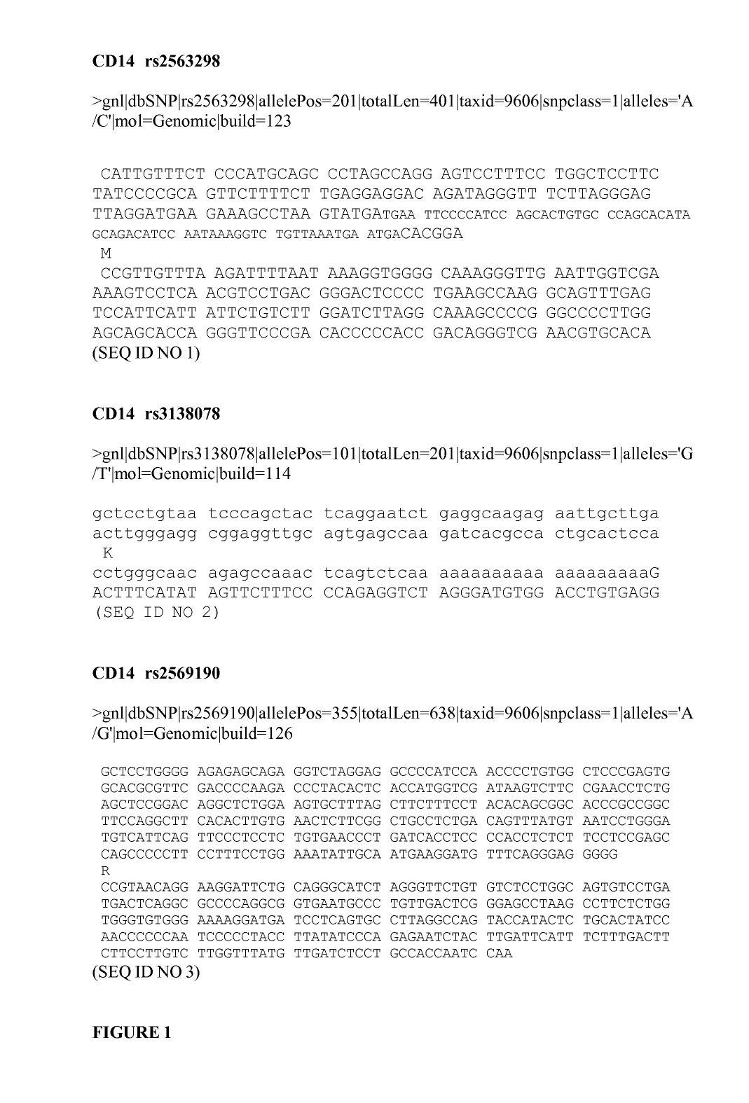

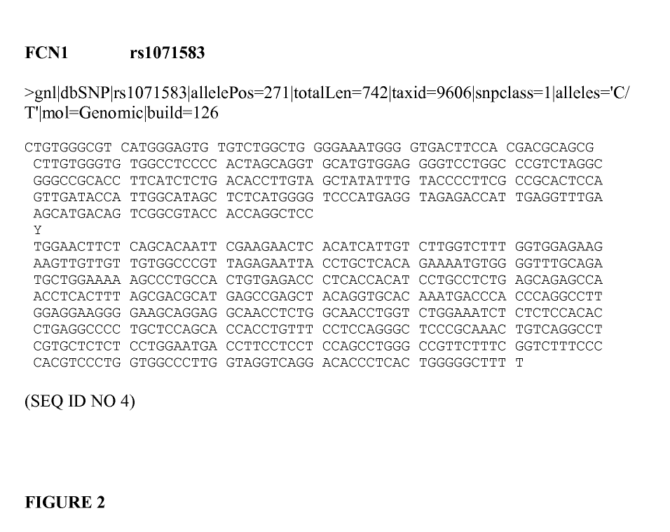

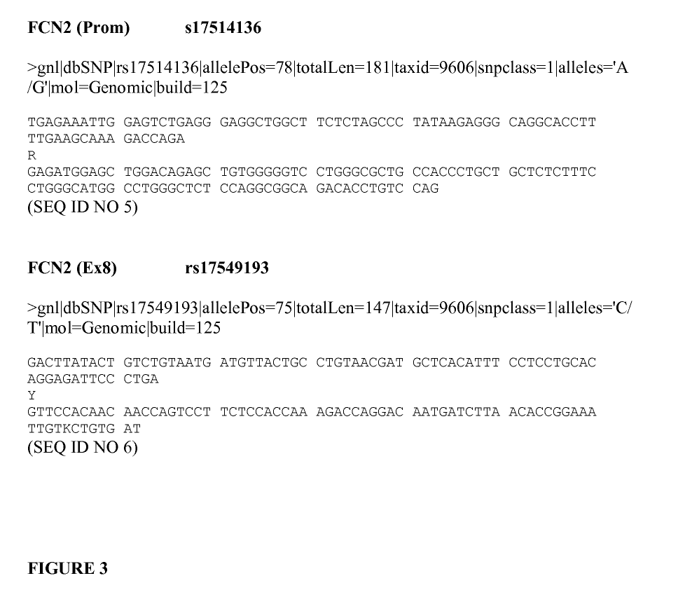

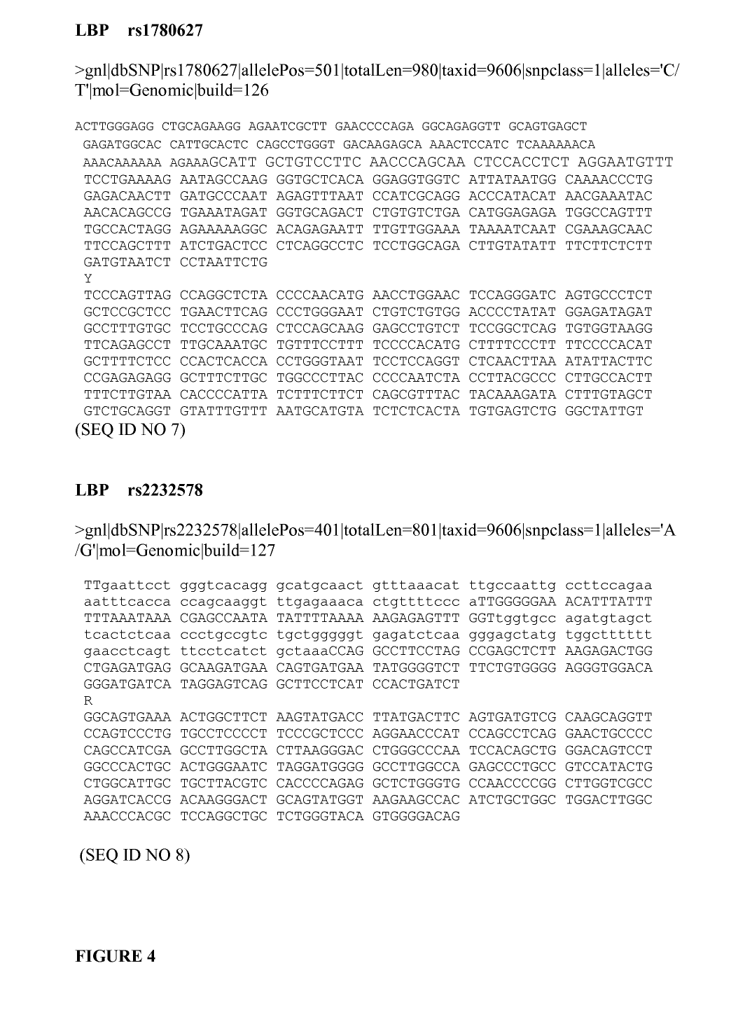

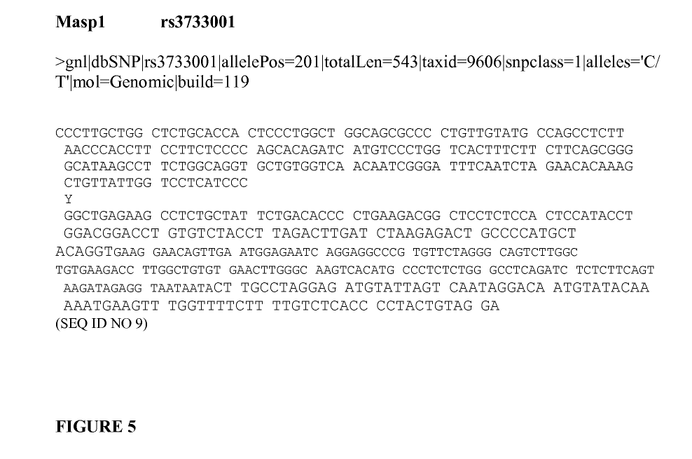

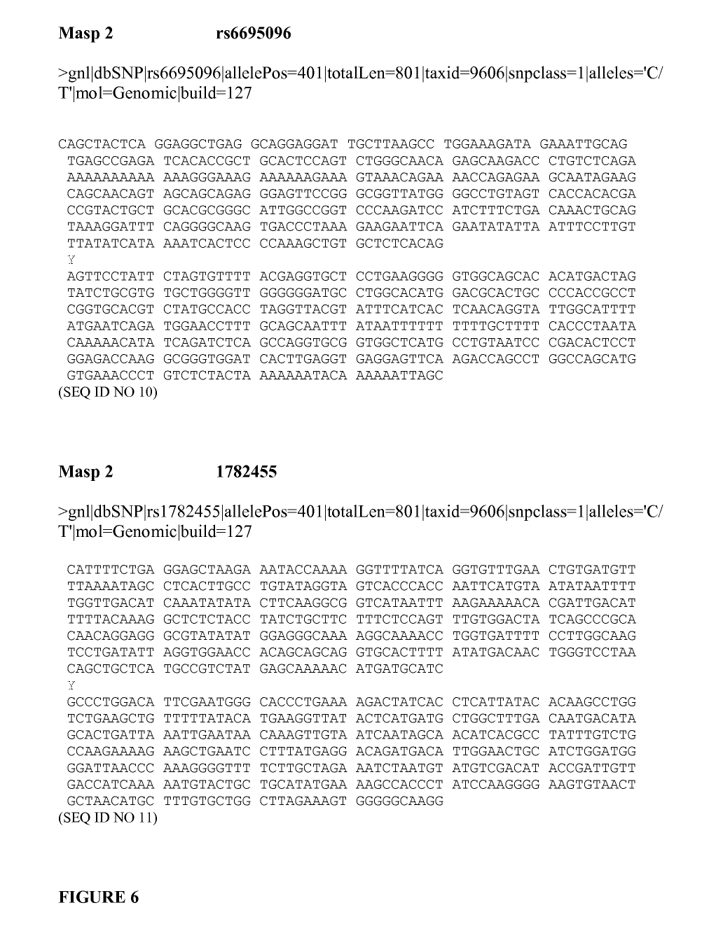

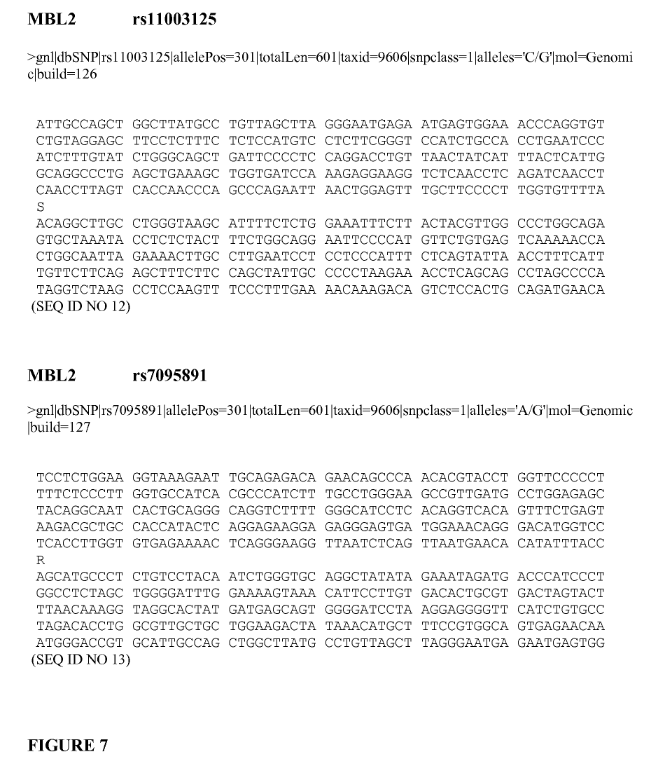

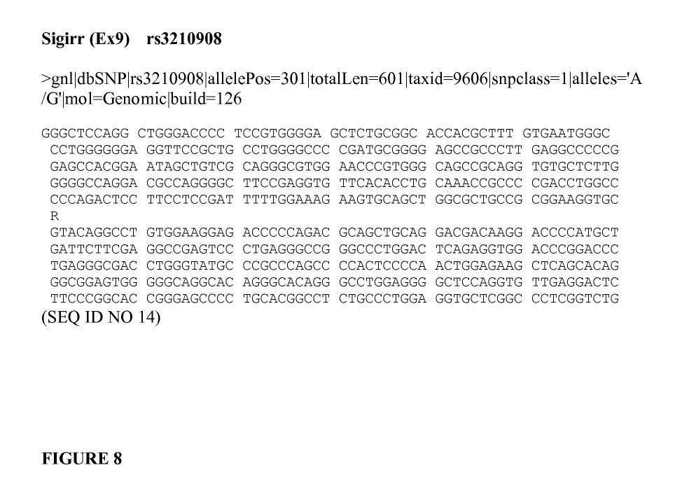

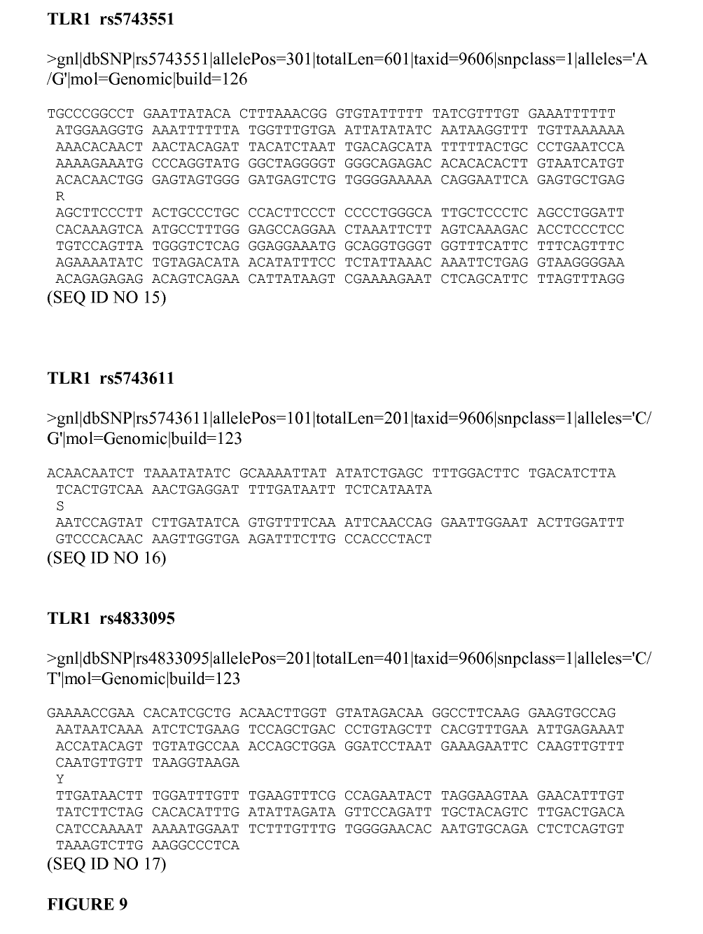

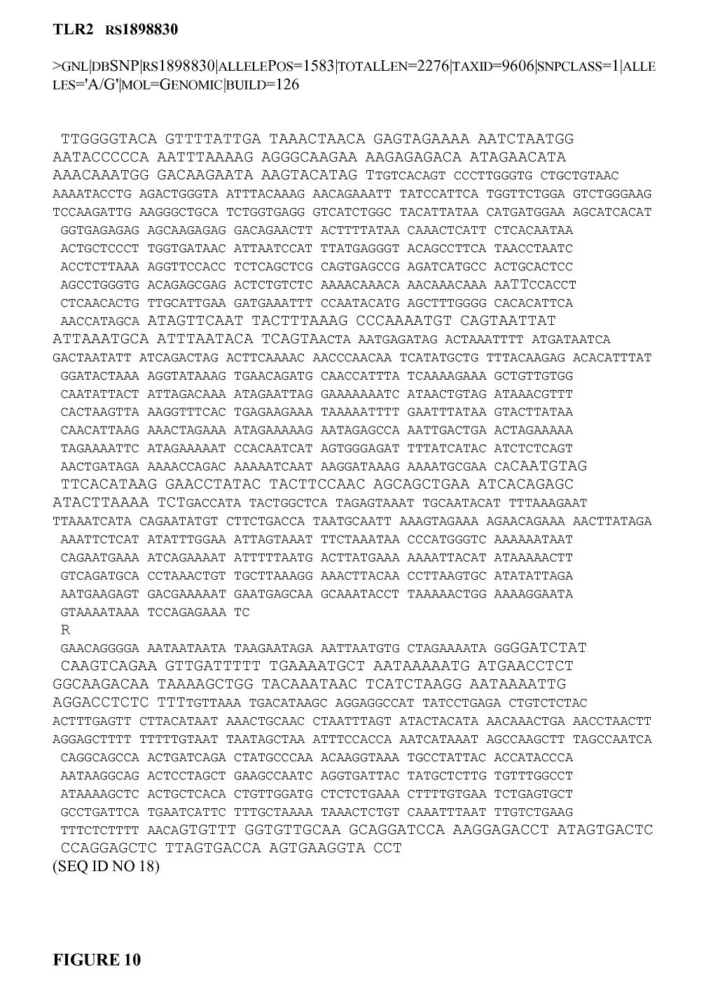

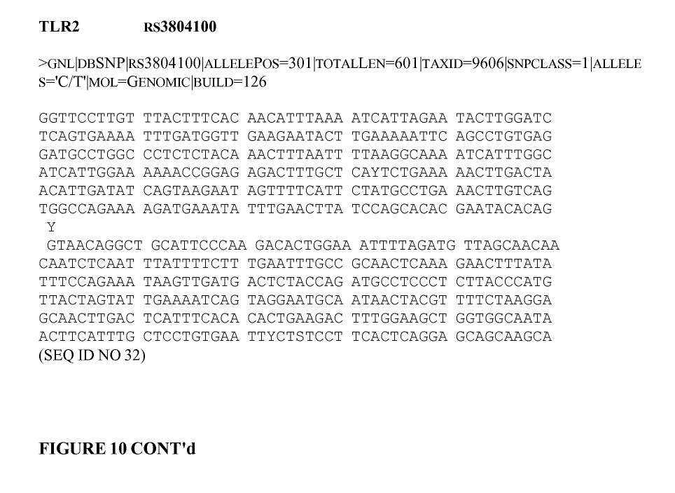

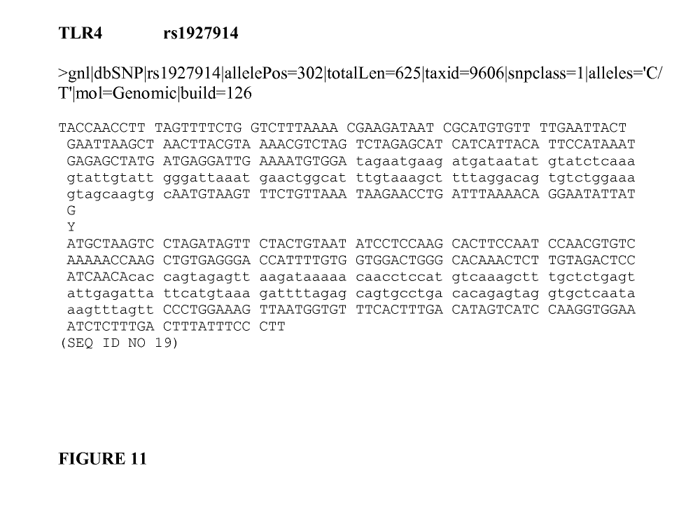

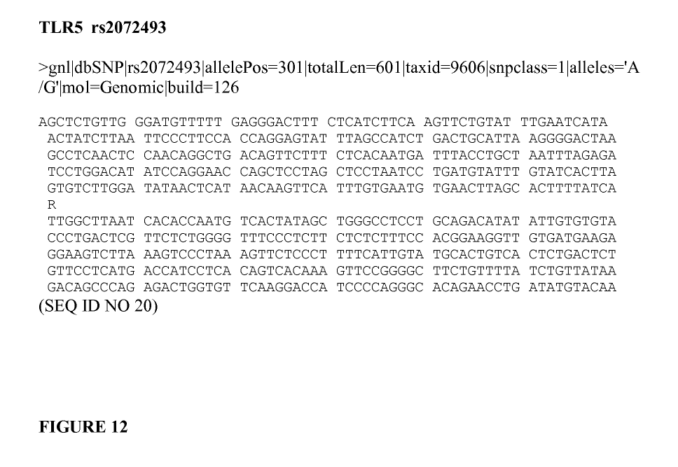

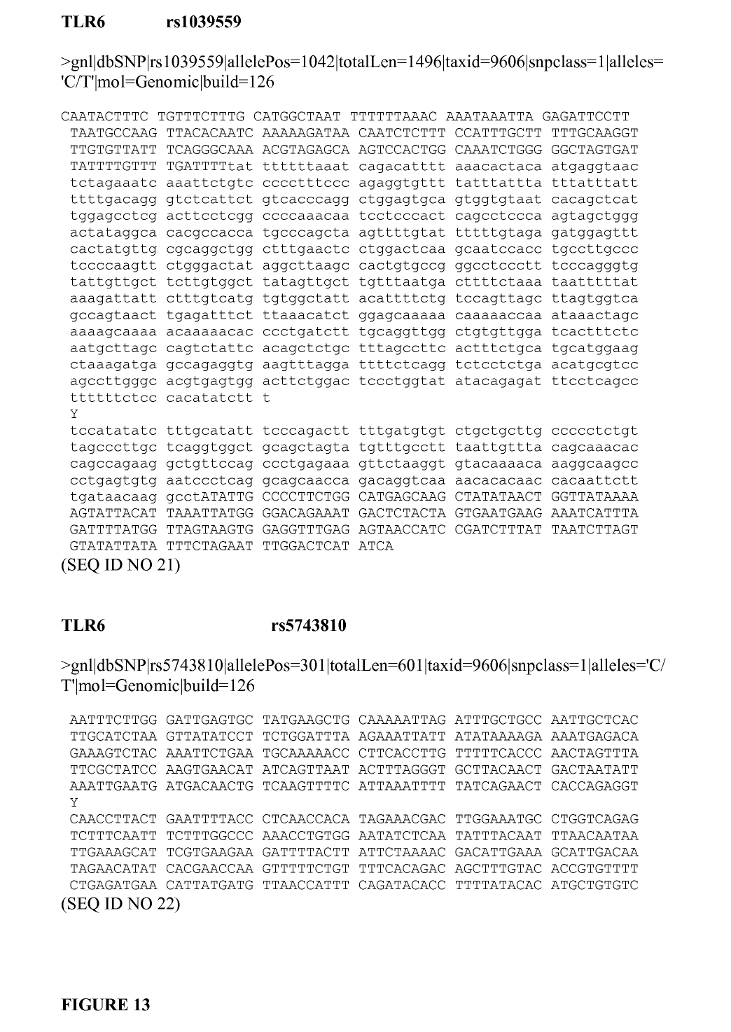

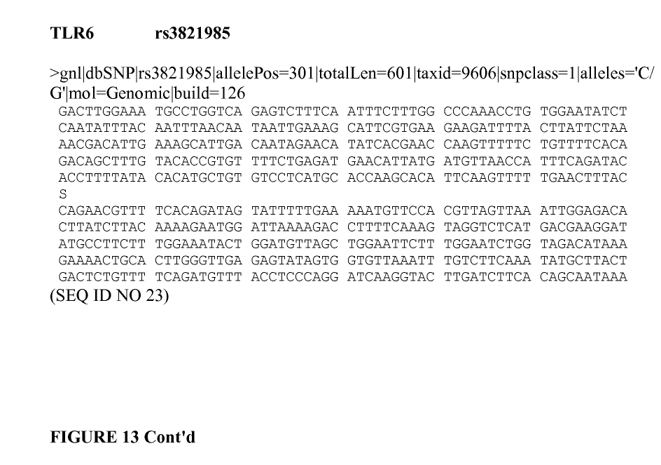

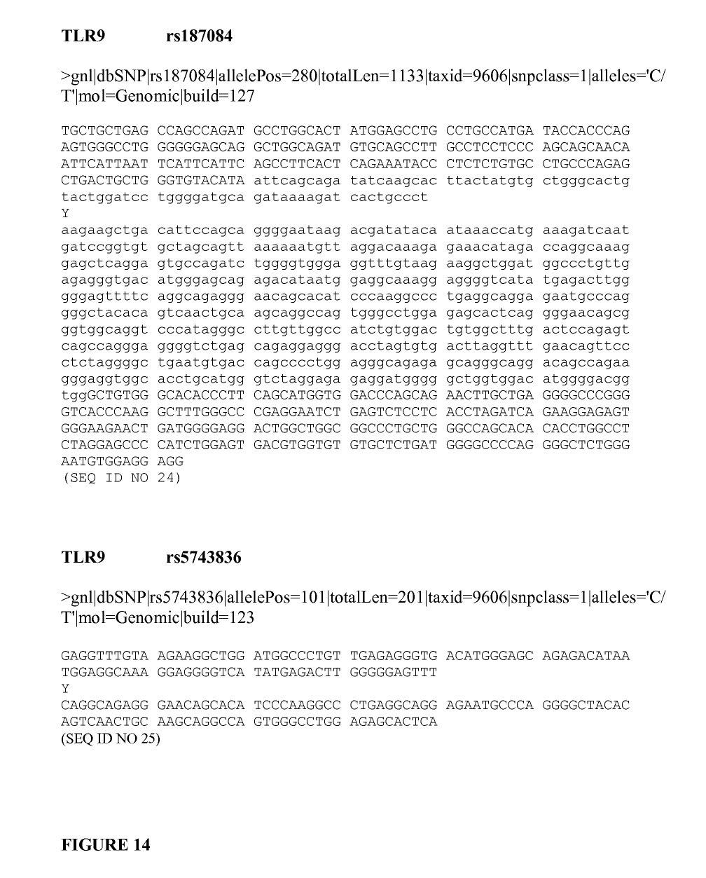

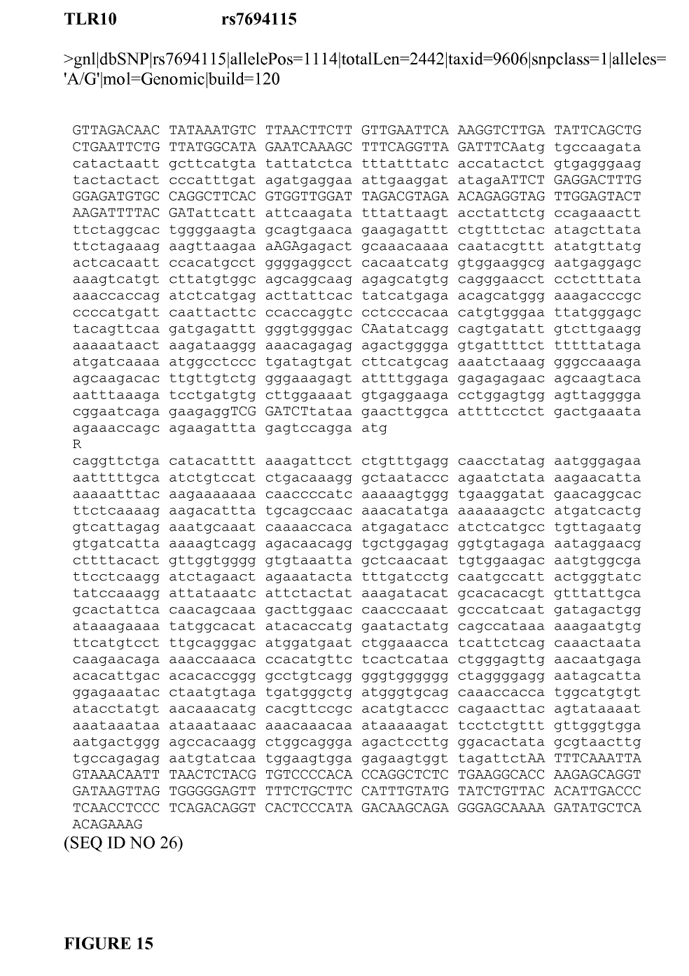

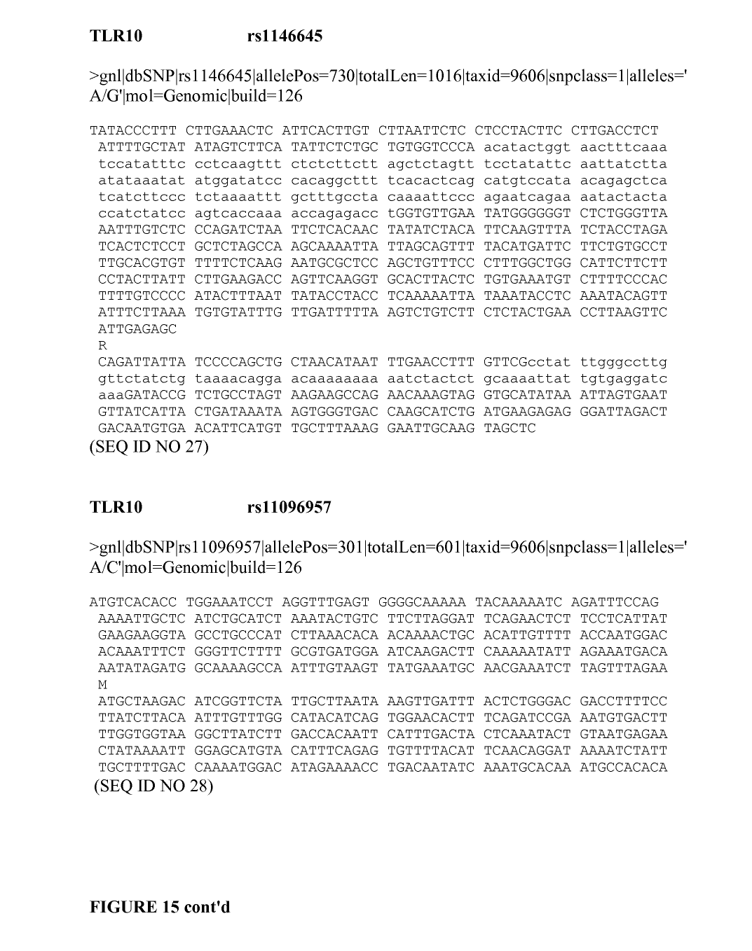

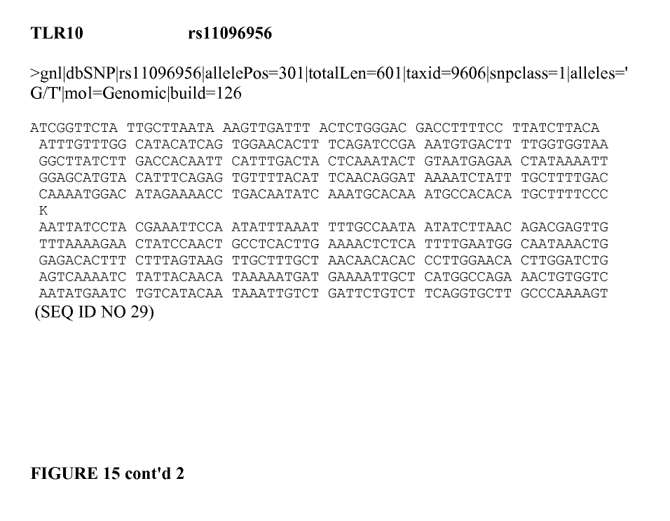

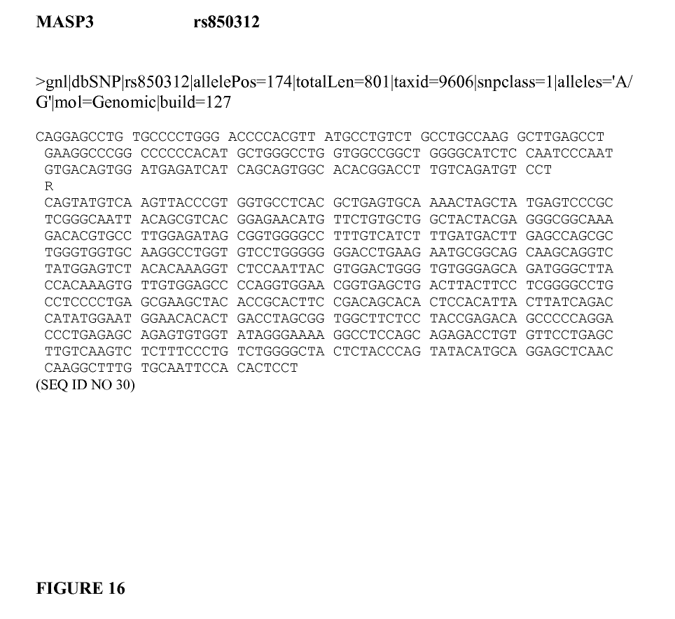

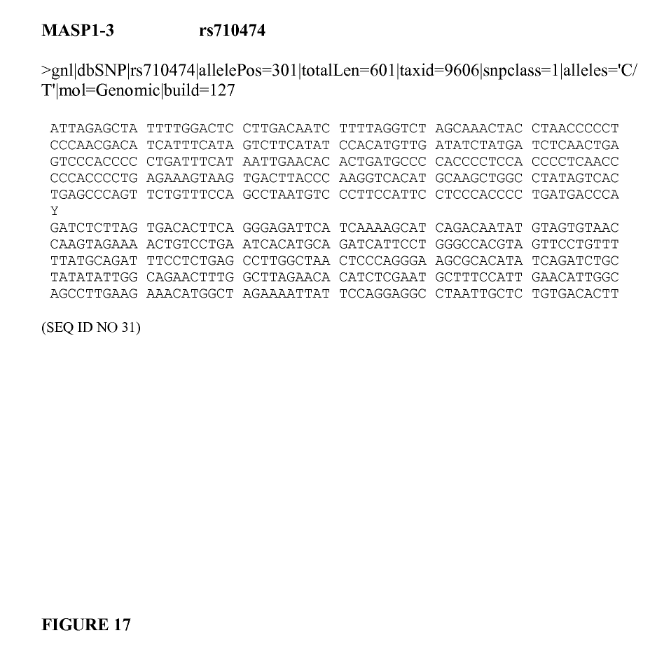

Furthermore, the relevant part of the genomic DNA of the gene comprising the nucleic acid variant as given in Table 2 is provided in FIGS. 1-17. Accordingly, in a specific embodiment, the polymorphism or nucleic acid variant is detected in the specified gene comprising said fragment of the gene, i.e. comprising the sequence as given by SEQ ID NO 1 to 31, respectively.

In a further embodiment, the methods of the invention comprise the step of determining whether the one or more nucleic acid variants in the gene are present in 0, 1 or 2 copies, more particularly whether a nucleic acid variant in the gene is present in one or in both alleles. Thus, according to this embodiment it is determined not only whether an allele carrying the nucleic acid variation is present or absent, but if it is present, it is also determined whether there is only one allele carrying the nucleic acid variant (heterozygous presence) or whether both alleles carry the nucleic acid variant (homozygous presence). Either heterozygous or homozygous presence of the at least one nucleic acid variant is indicative of the risk for impairment of the lung function and/or the risk for colonization with an infectious agent. This is shown in Tables 4-10.

According to yet a further embodiment, the methods of the invention comprise determining the presence of at least two genotypes. More specific, the presence of 0, 1 or 2 alleles in a single gene or in two different genes is linked to the development of impaired lung function and/or to susceptibility for colonization with P. aeruginosa or S. aureus.

In a more specific embodiment, the methods of the invention comprise determining the presence of a combination of at least two SNPs as given in Tables 5, 6, 8 and 10.

Different analytical procedures suitable for the detection of the presence or absence of the nucleic acid variants mentioned herein are known in the art. Nucleic acid from any nucleated cell can be used as the starting point for such assay techniques and may be isolated according to standard nucleic acid preparation procedures well known to those of skill in the art. Many current methods for the detection of allelic variation are reviewed by Nollau et al. (1997), Gut (2001), and in standard textbooks, for example “Laboratory Protocols for Mutation Detection”, Ed. by U. Landegren, Oxford University Press, 1996 and “PCR”, 2nd Edition” by Newton & Graham, BIOS Scientific Publishers Limited, 1997.

The step of determining the presence of a nucleic acid variant in a gene in the methods of the present invention can be carried out in vivo or in vitro. Most typically, however, detection of nucleic acid variants in the genes are performed in vitro in a biological sample obtained from the subject.

Typically, a nucleic acid comprising a sequence of interest can be obtained from a biological sample, more particularly from a sample comprising DNA (e.g. gDNA or cDNA) or RNA (e.g. mRNA). Release, concentration and isolation of the nucleic acids from the sample can be done by any method known in the art. Currently, various commercial kits are available such as the QIAamp DNA Blood Kit from Qiagen (Hilden, Germany) for the isolation of nucleic acids from blood samples, or the ‘High pure PCR Template Preparation Kit’ (Roche Diagnostics, Basel, Switzerland) or the DNA purification kits (PureGene, Gentra, Minneapolis, US). Other, well-known procedures for the isolation of DNA or RNA from a biological sample are also available (Sambrook et al., Cold Spring Harbor Laboratory Press 1989, Cold Spring Harbor, N.Y., USA; Ausubel et al., Current Protocols in Molecular Biology 2003, John Wiley & Sons).

If needed, e.g. when the quantity of the nucleic acid is low or insufficient for the assessment, the nucleic acid of interest may be amplified. Such amplification procedures can be accomplished by those methods known in the art, including, for example, the polymerase chain reaction (PCR), ligase chain reaction (LCR), nucleic acid sequence-based amplification (NASBA), strand displacement amplification, rolling circle amplification, T7-polymerase amplification, and reverse transcription polymerase reaction (RT-PCR).

Accordingly the methods of the present invention optionally comprise the steps of isolating nucleic acids from the sample and/or an amplification step.

Numerous methods for detecting a single nucleotide anomaly in nucleic acid sequences are well-known in the art. The present invention is not limited by any particular method used to detect the target sequences disclosed herein. Examples of such methods are described by Gut (2001) and Syvänen (2001), and include, but are not limited to, hybridization methods such as reverse dot blot, line probe assay (LiPA), GeneChip™ microarrays, dynamic allele-specific hybridization (DASH), peptide nucleic acid (PNA) and locked nucleic acid (LNA) probes, TaqMan™ (5′ nuclease assay), and molecular beacons; allele-specific PCR methods such as intercalating dye, FRET primers, and Alphascreen™; primer extension methods such as ARMS (amplification refractory mutation system), kinetic or real-time PCR, SNPstream™, Genetic Bit Analysis™ (GBA), multiplex minisequencing, SnaPshot™, Pyrosequencing™, MassEXTEND™, MassArray™, GOOD assay, microarray minisequencing, APEX (arrayed primer extension), sequence specific priming (SSP), microarray primer extension, Tag arrays, coded microspheres, template-directed incorporation (TDI), fluorescence polarization; oligonucleotide ligation methods such as colorimetric OLA (oligonucleotide ligation assay), sequence-coded OLA, microarray ligation, ligase chain reaction, padlock probes, and rolling circle amplification; endonuclease cleavage methods such as restriction site analysis (RFLP) and Invader™ assay. More specific assays that can be used are LiPA, DoPA and a microarray.

In a particular embodiment, the detection of the presence or absence of a nucleic acid variant is determined by DNA or RNA hybridization, sequencing, PCR, primer extension, multiplex ligation-dependent probe amplification (MLPA), oligonucleotide ligation assay (OLA) or restriction site analysis.

Another aspect of the invention is the use of the present methods to identify the most effective treatment in order to prevent rapid decline of lung function and worsening of the disease. Determining the presence of specific genotypes as described herein may lead to a considerable decrease in morbidity. The methods of the invention thus provide a tool for improved risk assessment and may help to determine the most appropriate, “personalized” therapy.

Another aspect of the invention relates to a kit for use in the methods as described herein. More specific the present invention encompasses a kit for identifying a subject at risk of an impaired lung function and/or the susceptibility for colonization with an infectious agent, comprising one or more reagents for detecting the presence (or, where appropriate absence) of one, two or more nucleic acid variants in a gene or a combination of genes. More particular, the kits of the present invention provide the tools for the detection of one or more nucleic acid variants in the genes as given in Table 2, and more specific the nucleic acid variants as given in Tables 4, 7, or 9, or, the combinations as given in Tables 5, 6, 8, or 10.

In a specific embodiment, the invention relates to a kit for identifying (1) a subject at risk of an impaired lung function and/or (2) a subject susceptible for colonization with an infectious agent. The kit comprises one or more reagents for detecting the presence (or, where appropriate absence) of at least two nucleic acid variants selected from the group as given in Table 2. More particularly the kits of the present invention provide the tools for the detection of the specific nucleic acid variant as provided in Tables 4, 7, and 9, and/or the specific combination of at least two nucleic acid variants as provided in Tables 5, 6, 8, and 10.

Although different reagents or tools suitable for detecting the presence of a nucleic acid variant in a gene can be envisaged, in a particular embodiment they will include an oligonucleotide probe suitable for detection of a target polynucleic acid and/or an oligonucleotide pair suitable for amplification of a target polynucleic acid. Specific embodiments of the kits of the present invention comprise an oligonucleotide probe suitable for detection of a sequence within the gene and/or an oligonucleotide pair suitable for amplification of a sequence within a polynucleic acid.

Oligonucleotides for use in the kits or methods of the present invention typically are isolated nucleic acid molecules comprising at least 8 nucleotides and specifically hybridizing with a target nucleic acid sequence, e.g. the wild type or variant sequence of the gene including the position of the nucleic acid variant, or the complementary thereof.

More particularly, the oligonucleotide comprises at least 9, 10, 11, 12, 13, 14 or 15 nucleotides and up to 40, 30, 25, 24, 23, 22, 21, or 20 nucleotides. The oligonucleotides can be used as a primer or probe. In general such primers or probes will comprise nucleotide sequences entirely complementary to the corresponding target sequence, e.g. a wild type or variant locus in the target gene. Of course, specific length and sequence of the probes and primers will depend on the complexity of the required nucleic acid target, as well as on the reaction conditions such as temperature and ionic strength. Preferably, the primers or probes will amplify or hybridize with the sequence characterized by SEQ ID NOs 1-32.

An oligonucleotide primer (or primer pair) designed to specifically recognize and amplify either a wild type or variant allele at a locus is referred to as an allele specific primer (or primer pair). The same applies for an allele specific probe, i.e. an oligonucleotide probe that specifically hybridizes to either a wild type or variant allele.

For the detection of specific alleles, or for the detection of specific nucleic acid variants, the hybridization conditions are to be stringent as known in the art. “Stringent” refers to conditions under which a nucleotide sequence will no longer bind to unrelated or non-specific sequences. For example, high temperature and lower salt increases stringency such that non-specific binding or binding with low melting temperature will be prevented or dissolved. (Meinkoth J et al., 1984).

The primers or probes may carry one or more labels to facilitate detection. The nature of the label is not critical to the invention and may be fluorescent, chemiluminescent, enzymatic, radioactive, chemical or other, provided it doesn't interfere with correct hybridization of the oligonucleotide.

Typically, the kits of the present invention will comprise one or more oligonucleotide primers or probes specific for the one or more allele(s) containing the nucleic acid variant(s). However, in another embodiment, it is envisaged that the kits for detection according to the methods of the present invention comprise one or more oligonucleotide primers or probes specific for the wild type allele, not containing the nucleic acid variant, whereby an indication of the presence of a wild type allele is indicative of the absence of a nucleic acid variant. In a further embodiment, the kits of the present invention comprise both oligonucleotide primers (or probes) specific for the “variant” and the “wild type” allele. The latter embodiment is particularly suited to determine the copy number of the variant alleles.

Accordingly, in specific embodiments, the one or more allele specific primers or probes for detecting the nucleic acid variants will typically comprise at least part of the nucleic acid sequence as identified in Table 1, e.g. as characterized by SEQ ID NO 1-32 respectively, or the complementary thereof, wherein the part of the nucleic acid sequence is envisaged to potentially comprise one or more nucleic acid variants. More specifically, kits are envisaged which comprise combinations of at least two primers or probes capable of hybridizing to and/or amplifying the region comprising the SNP's indicated in Table 2, wherein the designated positions either have the wild type nucleotides or nucleic acid variants thereof.

Accordingly, the present invention also provides kits comprising two or more allele specific primers and/or one or more oligonucleotide probes for detecting the presence of one or at least two, i.e. 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38, nucleic acid variants. Preferably, the one, two or more nucleic acid variants are selected from the group as given in Table 2. More preferably, the single nucleic acid variants are selected from Tables 4, 7 and 9. Even more preferably, the two or more nucleic acid variants are those combinations as specifically given in Tables 5, 6, 8, or 10.

Apart from the one or more specific reagents for detecting the presence of markers for the prognosis of lung function and/or severity of infection with microorganisms described above, the kits of the invention optionally contain a variety of other reagents, e.g. depending on the detection procedure. The following is a non-exhaustive list of reagents that may be part of the kit's contents, the person skilled in the art will understand that this merely is illustrative of the possibilities: an agent for denaturing nucleic acids, an enzyme capable of modifying a double stranded or single stranded nucleic acid molecule, a hybridization buffer, or components necessary for producing a hybridization buffer, a wash solution, or components necessary for producing a wash solution, a means for detecting partially or completely denatured polynucleic acids, a means for detecting hybrids formed in the preceding hybridization, a means for detecting enzymatic modifications of nucleic acids, a means for attaching an oligonucleotide to a known location on a solid support, a labelled antibody, and/or a means for attaching an antigen to a known location on a solid support.

In a preferred embodiment of the kit, the means for determining the risk for impaired lung function, or the means for determining the susceptibility for colonization with an infectious agent include a table, a chart, or similar, generally referred to as “a predisposition risk algorithm”, indicating the SNPs, SNP combinations or haplotypes as described herein that confer said risk (increased or decreased) or susceptibility. As used herein, the term “chart” refers to graphical presentation, visual aid, diagram, plan, graph, sheet, map or the like including the relevant information. The determination of the risk can be performed manually or with the use of a computer.

Other arrangements of the methods and tools embodying the invention will be obvious for those skilled in the art.

It is to be understood that although preferred embodiments, specific constructions and configurations, as well as materials, have been discussed herein for the methods and tools according to the present invention, various changes or modifications in form and detail may be made without departing from the scope and spirit of this invention.

The present invention is illustrated by the following Examples, which should not be understood to limit the scope of the invention to the specific embodiments therein.

### EXAMPLES

### Statistical Analysis

For the present examples, the statistical analysis of the data is based on the determination of odds ratios (OR) using standard procedures. An odds ratio is calculated by dividing the odds in the treated or exposed (case) group by the odds in the control group. The odds of an event are calculated as the number of events divided by the number of non-events. If the odds of an event are greater than one the event is more likely to happen than not (the odds of an event that is certain to happen are infinite); if the odds are less than one the chances are that the event won't happen (the odds of an impossible event are zero). In the present examples, the strength of association was reported as odds ratios (OR) (with 95% lower (LCL) and upper (UCL) confidence limit), indicating the factor by which the risk of developing a disorder or disease is increased (OR>1), or indicating the factor for a protective effect on the risk of developing a disorder or disease (OR<1).

The 95% confidence interval (95% CI) is the range of numerical values in which we can be confident (to a computed probability, here 95%) that the population value being estimated will be found. Confidence intervals indicate the strength of evidence; where confidence intervals are wide, they indicate less precise estimates of effect. The larger the trial's sample size, the larger the number of outcome events and the greater becomes the confidence that the true relative risk reduction is close to the value stated. Thus the confidence intervals get narrower and “precision” is increased. To confidently accept a calculated OR as reliable, important or clinically significant, the lower boundary of the confidence interval, or lower confidence limit, should be >1 if the OR>1, or the upper boundary of the confidence interval should be <1 if the OR<1.

### Patient Samples

Genomic DNA of 101 (6-44 years old, whereby 68 adults (>16 years)) cystic fibrosis patients was obtained for genetic analysis. Proven diagnosis of CF was based on CFTR genotyping (available on all patients) and clinical phenotype. Of these patients the Forced expiratory volume in 1 second (FEV1%) (average value from year 1 until year 6), and colonisation with Pseudomonas aeruginosa and Staphylococcus was determined.

For each patient, informed consent to participate in the study is available.

### Materials & Methods

The relevant areas of the genes listed in Table 3, were amplified and hybridised to (i) either LiPA strips for MBL2 according to the protocol described by the manufacturer of the kit (INNO-LiPA MBL2, Innogenetics NV), (ii) or DoPA (Dot Probe Assay) strips for the remaining genes. The protocol for the DoPA strips is similar to the protocol for the MBL2 LiPA strips.

TABLE 3

| | # genes | # snp's | # PCR | LiPA/DoPA |
|---|---|---|---|---|
| MBL | MBL | 6 | 1 duplex | INNO-LiPA |
| Secreted | MASP ⅓ | 3 | 1 multiplex | 1 DOPA |
| | MASP 2 | 6 | | |
| | FCN1 | 2 | | |
| | FCN2 | 4 | | |
| | FCN3 | 1 | | |
| | C1QR1 | 2 | | |
| Signaling 1 | LBP | 6 | 1 multiplex | 1 DOPA |
| | CD14 | 4 | | |
| | LY96 | 3 | | |
| | SIGIRR | 3 | | |
| | CARD15 | 3 | 1 single | |
| Signaling 2 | TLR1 | 4 | 1 multiplex | 1 DOPA |
| | TLR2 | 4 | | |
| | TLR3 | 3 | | |
| | TLR4 | 3 | | |
| | TLR5 | 4 | | |
| | TLR6 | 4 | 1 multiplex | |
| | TLR7 | 3 | | |
| | TLR8 | 5 | | |
| | TLR9 | 3 | | |
| | TLR10 | 5 | | |
| Total | 22 | 81 | 6 | 4 |

In general, the relevant regions were amplified using biotinylated oligonucleotides. The polymorphisms were detected by use of a reverse hybridisation method with probes designed to recognize the polymorphisms. After stringent wash at 56° C., hybridised probes were incubated with a streptavidine-alkaline phosphatase conjugate. The presence of a hybridised probe was revealed using NBIT/BCIP color development. Details on the reverse hybridisation are described in Stuyver et al. (1996), Stuyver et al. (1997) and Van Geyt et al. (1998).

### SNP Analysis and Results

From the hybridization results for each locus the genotype was determined and odds ratios for individual SNP and combinations of two SNP's were calculated.

From these, the most valuable SNPs or SNP combinations were selected using the following criteria: OR≧3 or OR≦0.4 and the number of patients with that particular SNP combination is ≧15 in the study population. The correlations found are described below.

Correlation with Lung Function

Lung function in cystic fibrosis patients and in particular the decline of it is an important measure for the follow up and treatment of the disease. From age six, approximately, the lung function, expressed as FEV1% can be accurately measured. The decline (deltaFEV1) over the years is indicative for lung tissue damage and hence the progression of the disease. In this study, the mean FEV1% value over 5 years was correlated with the SNPs analysed. In addition, the decline was measured over a five year period and correlated with the SNPs analysed.

The CF patient cohort was subdivided in two groups;
for FEV1%:
   (i) more than 70% (acceptable/good lung function), and
   (ii) less than 70% (impaired/bad lung function);
for deltaFEV1:
   (i) less than 14% decline over a 5-year period (minor decline of lung function), and
   (ii) more than 14% decline over a 5-year period (strong decline of lung function).

In adults (>16 year) 4 single SNPs that correlated significantly with the FEV1% were found and there were 5 SNP combinations that matched the criteria (Table 4 and Table 5). From Tables 4 and 5, it can be concluded that SNPs or a SNP combination with an OR greater than 3 indicate a high risk for developing a lung function with a FEV1% value of less than 70%, i.e. a risk for impaired lung function. SNPs or SNP combinations with an OR less than 0.4 indicate a low risk for developing a lung function with a FEV1% value of less than 70%, i.e. a low risk for impaired lung function.

For deltaFEV1 single SNPs did not reveal significant ORs; but in the total CF cohort as well as in adults 4 SNP combinations showed significant ORs (Table 6). From Table 6, it can be concluded that a SNP combination with an OR greater than 3 indicate a high risk for a rapid or strong decline of the lung function (deltaFEV1 more than 14%). SNP combinations with an OR less than 0.4 indicate low risk for a decline of the lung function (deltaFEV1 less than 14%) and thus milder clinical disease.

TABLE 4
Overview OR and 95% confidence interval for SNPs that correlated
significantly with the FEV1% (greater or less than 70%)

| SNP | | | OR (95% Cl) |
|---|---|---|---|
| CD14 (prom) | rs3138078 | GT | 3,11(1,08-8,93) |
| CD14 (Ex2) | rs2563298 | GG | 0,34(0,12-0,94) |
| TLR10 (exon3) | rs11096956 | GG | 3,17(1,09-10,09) |
| TLR10 (promoter) | rs11466645 | TT | 3,47(1,15-10,53) |

TABLE 5
Overview OR and 95% confidence interval for SNP combinations that
correlated significantly with the FEV1% (greater or less than 70%).

| SNP 1 | | | SNP 2 | | | OR (95% CI) |
|---|---|---|---|---|---|---|
| MASP3 | rs850312 | GA | Masp2 | rs6695096 | AA | 0.26 |
| | | | | | | (0.08-0.82) |
| MASP3 | rs850312 | GG | TLR9 | rs5743836 | TT | 5.55 |
| | | | | | | (1.67-18.41) |
| MASP2 | rs6695096 | AA | CD14 | rs3138078 | GG | 0.28 |
| | | | | | | (0.10-0.83) |
| FCN1 | rs1071583 | AG | TLR5 | rs2072493 | AA | 6.19 |
| | | | | | | (1.72-22.24) |
| CD14 | rs3138078 | GT | TLR10 | rs1146645 | TT | 6.19 |
| | | | | | | (1.72-22.24) |

TABLE 6
Overview OR and 95% confidence interval for SNP combinations that
correlated significantly with the DeltaFEV1 (decline greater or less than 14%)
over 5 years.

| | | | | | | OR (95% CI) | |
|---|---|---|---|---|---|---|---|
| SNP 1 | | | SNP 2 | | | Total cohort | Adults only |
| MASP1-3 | rs710474 | CC | FCN2 | −4A > G | AA | 0.36 (0.13-1.00) | 0.21 (0.06-0.75) |
| MASP1-3 | rs710474 | CC | TLR5 | rs2072493 | AA | 0.3 (0.11-0.82) | 0.28 (0.09-0.82) |
| MASP2 | rs6695096 | AA | TLR2 | rs1898830 | AG | 7.19 (1.9-27.21) | 6.53 (1.66-25.78) |
| MASP2 | rs6695096 | AA | TLR4 | rs1927914 | GA | 4.91 (1.6-15.04) | 5.33 (1.53-18.59) |

Correlation with Pseudomonas Aeruginosa Colonisation.

Pseudomonas aeruginosa is a opportunistic pathogen seldom encountered as an infectious agent in healthy or non-immunocompromised individuals. However in CF patients Pseudomonas aeruginosa infections are frequent and eventually lead to colonisation. In general colonised patients have increased lung dysfunction and a bad prognosis with many episodes of antibiotic treatment (often intravenously) and hospitalization. Therefore it is mandatory that risk for severe infection and in particular colonisation with Pseudomonas aeruginosa can be predicted in order to be able to prevent it.

As shown in Table 7, three signal SNPs produced significant OR although significance was either found in the total cohort only (Masp3) or in adults only (LBP & TLR1). In Table 8, 16 SNP combinations that match the criteria are listed. Six combinations have significant ORs in both the total cohort and in adults.

In particular LBP and TLR SNPs and combinations with these show pronounced values (ORs up to 7).

From Table 7 and 8 it can be concluded that SNPs or a SNP combination with an OR greater than 3 indicate a high susceptibility for colonisation with P. aeruginosa. SNPs or a SNP combination with an OR less than 0.4 indicate a low susceptibility for colonisation with P. aeruginosa.

TABLE 7
Overview OR and 95% confidence interval for SNPs that correlated
significantly with Pseudomonas aeruginosa colonisation.

| | | | OR (95% Cl) | |
|---|---|---|---|---|
| SNP | | | Total cohort | Adults only |
| LBP | rs1780627 | TC | | 4,03(1,40-11,66) |
| Masp3 | rs850312 | GG | 0,38(0,16-0,90) | |
| TLR1 | rs4833095 | AA | | 0,34(0,12-0,93) |

TABLE 8
Overview OR and 95% confidence interval for SNP combinations that
correlated significantly with Pseudomonas aeruginosa colonisation.

| | | | | | | OR (95% CI) | |
|---|---|---|---|---|---|---|---|
| SNP 1 | | | SNP 2 | | | Total cohort | Adults only |
| LBP | rs2232578 | AA | LBP | rs1780627 | TC | 3.28 (1.15-9.31) | 6.26 (1.62-24.13) |
| LBP | rs1780627 | TC | TLR6 | rs3821985 | CG | 3.58 (1.27-10.13) | 4.07 (1.19-13.94) |
| LBP | rs1780627 | CC | TLR10 | rs1146645 | TT | 0.35 (0.12-0.96) | 0.24 (0.07-0.80) |
| MBL2 | +4C > T | CC | LBP | rs1780627 | TC | 3.28 (1.15-9.31) | 7.21 (1.49-34.81) |
| MBL2 | −550G > C | CC | TLR6 | rs3821985 | CG | 3.35 (1.10-10.20) | |
| Masp3 | rs850312 | GG | TLR1 | rs5743551 | AA | 0.19 (0.07-0.55) | 0.2 (0.06-0.68) |
| Masp3 | rs850312 | GA | TLR6 | rs1039559 | CT | 3.68 (1.22-11.15) | |
| Masp1 | rs3733001 | GG | TLR6 | rs5743810 | TC | | 0.22 (0.07-0.67) |
| MASP2 | rs6695096 | AA | LBP | rs1780627 | TC | | 4.97 (1.46-16.93) |
| FCN2 | −4AtoG | AG | TLR1 | rs5743551 | AA | | 0.25 (0.08-0.79) |
| FCN2 | −4AtoG | AG | TLR10 | rs1146645 | TT | | 0.30 (0.10-0.89) |
| CD14 | rs2563298 | GG | TLR1 | rs4833095 | AG | 3.28 (1.15-9.31) | |
| TLR1 | rs4833095 | AG | TLR6 | rs3821985 | CG | 3.58 (1.27-10.13) | 6.26 (1.62-24.13) |
| TLR1 | rs4833095 | AA | TLR6 | rs3821985 | CG | | 0.22 (0.07-0.67) |
| TLR1 | rs4833095 | AA | TLR10 | rs1146645 | TT | | 0.34 (0.12-0.93) |
| TLR2 | rs1898830 | AA | TLR6 | rs1039559 | CT | 3.28 (1.15-9.31) | |

Correlation with Staphylococcus Colonisation.

Staphylococcus species are ubiquitous bacteria usually harmless for human beings unless secondary conditions increases the susceptibility of the host. One such condition is Cystic fibrosis. Although in CF patients considered less implicating than Pseudomonas aeriginosa, Staphylococcus infections evolving to colonisation of the lung can cause complications and require adapted treatment (e.g. antibiotics).

Five single SNPs produced significant ORs, but only in the adult cohort (Table 9). In total 16 SNP combinations were identified with significant ORs in either the total or the adult population (Table 10). Six of them were significant in both groups analysed. The majority of the combinations involved MASP or FCN SNPs with TLR SNPs. From Table 9 and 10 it can be concluded that SNPs or a SNP combination with an OR greater than 3 indicate a high susceptibility for colonisation with Staphylococcus. SNPs or a SNP combination with an OR less than 0.4 indicate a low susceptibility for colonisation with Staphylococcus.

TABLE 9
Overview OR and 95% confidence interval for SNPs that
correlated significantly with Staphylococcus colonisation.

| | | | OR (95% Cl) |
|---|---|---|---|
| SNP | | | Adults only |
| Masp1-3 | rs710474 | TC | 0,32(0,11-0,94) |
| Masp2 | rs6695096 | AA | 0,14(0,03-0,67) |
| Masp2 | rs6695096 | GA | 7,22(1,50-34,86) |
| Masp2 | rs1782455 | CT | 7,92(1,64-38,18) |
| Sigirr | rs3210908 | AG | 3,61(1,18-11,08) |

TABLE 10
Overview OR and 95% confidence interval for SNP combinations that
correlated significantly with Staphylococcus colonisation.

| | | | | | | OR (95% CI) | |
|---|---|---|---|---|---|---|---|
| SNP 1 | | | SNP 2 | | | Total cohort | Adults only |
| Masp1-3 | rs710474 | CC | CD14 | rs2569190 | TC | 3.28 (1.02-10.49) | 12 (1.47-97.78) |
| Masp1-3 | rs710474 | TC | TLR5 | rs2072493 | AA | 0.30 (0.12-0.75) | 0.16 (0.05-0.52) |
| Masp1-3 | rs710474 | CC | TLR9 | rs187084 | CT | 3.28 (1.02-10.49) | |
| Masp1-3 | rs710474 | CC | TLR10 | rs7694115 | TC | | 4.56 (1.17-17.77) |
| MASP2 | rs6695096 | AA | TLR2 | rs1898830 | AG | | 0.24 (0.08-0.77) |
| MASP2 | rs6695096 | AA | TLR9 | rs187084 | TT | 0.34 (0.13-0.86) | 0.24 (0.08-0.77) |
| Masp2 | rs1782455 | CT | TLR1 | rs 5743611 | GG | 4.23 (1.33-13.44) | 15.2 (1.87-123.49) |
| Masp2 | rs1782455 | CT | CD14 | rs2569190 | TC | | 5.43 (1.12-26.39) |
| Masp2 | rs1782455 | CT | TLR9 | rs187084 | TT | 0.31 (0.12-0.79) | 0.19 (0.06-0.60) |
| FCN2 | −4AtoG | AG | TLR1 | rs5743551 | AA | 3.28 (1.02-10.49) | 4.67 (0.96-22.79) |
| FCN2 | −4AtoG | AG | TLR10 | rs11096956 | GG | 3.73 (1.17-11.89) | 12 (1.47-97.78) |
| FCN2 | −4AtoG | AG | TLR10 | rs1146645 | TT | 3.07 (1.05-8.97) | 5.98 (1.23-28.99) |
| FCN2 | T236M | CT | TLR10 | rs1146645 | TT | 3.27 (1.12-9.53) | 6.58 (1.36-31.80) |
| Sigirr | rs3210908 | AG | TLR1 | rs 5743611 | GG | 3.14 (1.15-8.61) | 5.48 (1.41-21.30) |

### REFERENCES

Den Dunnen J T and Antonarakis S E. Mutation nomenclature extensions and suggestions to describe complex mutations: A discussion. Hum. Mut. 2000; 15: 7-12. Hummelshoj et al. Hum Mol Genet. 2005; 14(12):1651-8.
Gut I. G. (2001) Automation in genotyping of single nucleotide polymorphisms. Hum. Mutat. 17: 475-492.
Meinkoth J and Wahl G, Analytical Biochemistry 138, 267-284 (1984).
Nollau P, Wagener C. (1997) Methods for detection of point mutations: performance and quality assessment. IFCC Scientific Division, Committee on Molecular Biology Techniques. Clin Chem., 43(7):1114-28.
Sambrook J., Fritsch E. and Maniatis T. (1989) Molecular cloning: a laboratory manual. Cold Spring Harbor Laboratory Press, Cold Spring Harbor, N.Y., USA.
Smith et al., Cell. 85, 229-236, 1996
Syvanen A. C. (2001) Accessing genetic variation: genotyping single nucleotide polymorphisms. Nat. Rev. Genet. 2: 930-942.
Stuyver L., Wyseur A., van Arnhem W., Hernandez F., Maertens G. (1996) A second generation line probe assay for hepatitis C virus. J. Clin. Microbiol. 34: 2259-2266.
Stuyver L., Wyseur A., Rombout A., Louwagie J., Scarcez T., Verhofstede C., Rimland D., Schinazi R. F., Rossau R. (1997) Line probe assay (LiPA) for the rapid detection of drug-selected mutations in the HIV-1 reverse transcriptase gene. Antimicrob. Agents Chemother. 41: 284-291.
Van Geyt C., De Gendt S., Rombaut A., Wyseur A., Maertens G., Rossau R., Stuyver L. (1998) A line probe assay for hepatitis B virus genotypes. In: R. F. Schinazi, J. P. Sommadossi, and H. Thomas (eds.). Therapies of viral hepatitis. International Medical Press, London, UK, pp. 139-145.

1. A method of determining the susceptibility for colonisation with an infectious agent, comprising determining the presence of at least one nucleic acid variant in a gene selected from the group consisting of the nucleic acid variants as given in Tables 7 and 9, and/or, determining the presence of a combination of nucleic acid variants in one or more genes, whereby the combination SNP1/SNP2 is selected from the group consisting of the nucleic acid variants as given in Tables 8 and 10.

2. A method of determining the risk for impaired lung function in a subject, comprising determining the presence of at least one nucleic acid variant in a gene elected from the group consisting of the nucleic acid variants as given in Table 4, and/or, determining the presence of a combination of nucleic acid variants in one or more genes, whereby the combination SNP1/SNP2 is selected from the group consisting of the nucleic acid variants as given in Tables 5 and 6.

3. The method according to claim 2, wherein the presence of one, and/or the combined presence of the at least two nucleic acid variants is indicative of the risk of deterioration of the pulmonary function and/or the risk of decline of the pulmonary function.

4. The method according to claim 1, wherein the presence of one, and/or the combined presence of the at least two nucleic acid variants is indicative of susceptibility for colonization with an infectious agent.

5. The method according to claim 4, wherein the infectious agent is a bacterium.

6. The method according to claim 5, wherein the bacterium is Pseudomonas aeruginosa and/or Staphylococcus aureus.

7. The method according to claim 1, wherein one or more of said at least one nucleic acid variant is a single nucleotide polymorphism (SNP).

8. The method according to claim 1, which comprises determining whether said nucleic acid variant in the gene is present in 0, 1 or 2 copies, wherein the heterozygous or homozygous presence of one or of the combination of the at least two nucleic acid variants is indicative of the predisposition of impaired lung function and/or susceptibility for colonization with an infectious agent.

9. The method according to claim 1, wherein the step of detecting the presence of the nucleic acid variant is performed by DNA or RNA hybridization, sequencing, PCR, primer extension, multiplex ligation-dependent probe amplification (MLPA), oligonucleotide ligation assay (OLA) and/or restriction site analysis.

10. The method according to claim 1, wherein the step of determining the presence of the nucleic acid variant is performed in vitro in a biological sample obtained from said subject.

11. A kit for determining the risk for impaired lung function in a subject comprising:
one or more reagents for detecting the presence of at least one nucleic acid variant in a gene selected from the group consisting of the nucleic acid variants as given in Table 4, and/or, for detecting the presence of a combination of nucleic acid variants in one or more genes, whereby the combination SNP1/SNP2 is selected from the group consisting of the nucleic acid variants as given in Tables 5 and 6.

12. A kit for determining the susceptibility for colonization with an infectious agent in a subject comprising: one or more reagents for detecting the presence of at least one nucleic acid variant in a gene selected from the group consisting of the nucleic acid variants as given in Tables 7 or 9, and/or, for detecting the presence of a combination of nucleic acid variants in one or more genes, whereby the combination SNP1/SNP2 is selected from the group consisting of the nucleic acid variants as given in Tables 8 and 10.

13. A diagnostic kit comprising:
one or more allele specific primers and/or one or more oligonucleotide probes for detecting the presence of at least one nucleic acid variant as given in Tables 4, 7, or 9, and/or,
one or more allele specific primers and/or one or more oligonucleotide probes for detecting the presence of a combination of nucleic acid variants in one or more genes, whereby the combination SNP1/SNP2 is selected from the group consisting of the nucleic acid variants as given in Tables 5, 6, 8, and 10.

14. A kit according to claim 11, further comprising a table indicating the link between the SNP or SNP combinations and the risk for impairment of lung function and/or susceptibility for colonization with an infectious agent.
